(19) United States
(12) Patent Application Publication
(10) Pub. No.: US 2025/0213719 A1
(43) Pub. Date: Jul. 3, 2025

(54) MAGNETIC NANOPARTICLES AND METHODS OF DRUG RELEASE

(71) Applicant: University of Notre Dame du Lac, South Bend, IN (US)

(72) Inventor: Prakash Daniel Nallathamby, South Bend, IN (US)

(21) Appl. No.: 18/849,978

(22) PCT Filed: May 1, 2023
(86) PCT No.: PCT/US2023/020529

§ 371 Date: Sep. 23, 2024

Related U.S. Application Data
(60) Provisional application No. 63/364,028, filed on May 2, 2022.

(51) Int. Cl. A61K 47/69; A61K 47/55; A61P 35/00; B82Y 5/00; B82Y 25/00; H01F 1/00; H01F 1/34

(52) U.S. Cl. CPC A61K 47/6923; A61K 47/551; A61P 35/00; H01F 1/0054; H01F 1/344; B82Y 5/00; B82Y 25/00

(57) ABSTRACT

Described herein are compositions, systems, and methods for targeted and controlled drug release. In some embodiments, the compositions, systems, and methods may comprise magnetoelectric silica nanoparticles for targeted and controlled release of chemotherapeutic drugs for cancer treatment. In some embodiments, an external magnetic field may be used to release one or more drugs from the magnetoelectric silica nanoparticles. The disclosed compositions, systems, and methods may improve drug targeting and reduce systemic drug toxicity.

### CROSS-REFERENCE TO RELATED APPLICATION(S)

This application claims priority to U.S. Provisional Patent Application No. 63/364,028, filed on May 2, 2022, which is incorporated by reference herein in its entirety.

### TECHNICAL FIELD

Described herein are compositions, systems, and methods for targeted and controlled drug release. In some embodiments, the compositions, systems, and methods may comprise magnetoelectric silica nanoparticles for targeted and controlled release of chemotherapeutic drugs for cancer treatment. In some embodiments, an external magnetic field may be used to release one or more drugs from the magnetoelectric silica nanoparticles. The disclosed compositions, systems, and methods may improve drug targeting and reduce systemic drug toxicity.

### BACKGROUND

The use of anthracyclines, such as doxorubicin (Dox) in cancer treatment, is limited by a number of side effects, which include the acute reversible toxicities of nausea, vomiting, stomatitis, and bone marrow suppression. The efficacy of anthracyclines in treating cancer is further limited by dose-dependent systemic toxicity (e.g., cardiotoxicity, neurotoxicity, vascular toxicity, etc.), with a cumulative dose >550 mg/m² causing an increase in the prevalence of heart failure and vascular damage. This progressive toxicity usually manifests after anthracycline therapy and may become apparent within one year of the completion of treatment (early onset) or many years after chemotherapy has been completed (late onset). The long-term organ toxicity caused by the anthracyclines includes, for example, vascular dysfunction and irreversible cardiomyocyte death, and therefore chronic reduced heart function. Recent studies of breast cancer survivors have also consistently shown changes in their cognitive function following chemotherapy, including memory loss, a tendency for lack of focus, and difficulty in performing simultaneous multiple tasks. These cognitive problems, collectively called somnolence or cognitive dysfunction, are also reported in cancer patients, especially breast cancer patients, undergoing Dox-based chemotherapy. Despite the numerous side-effects, some of which are chronic, anthracyclines such as Dox remain an important class of chemotherapeutic agents against solid tumors, which makes abandoning them not an option.

Another factor affecting anthracycline efficacy is that approximately 50% of Dox is eliminated from the body without any change in its structure, while the remainder of the drug is processed through three major metabolic pathways. Metabolism of anthracyclines occurs through hydroxylation, semiquinone formation, or deoxyaglycone formation, which can result in the formation of metabolites that either augment or suppress the anti-cancer properties of anthracyclines. Consequently, localizing Dox specifically to cancer cells will increase exposure of cancer cells to a larger cumulative dose while negating the off-target metabolism and systemic toxicity of the drug.

Magneto-electric nanoparticles (MENs) may be suitable candidates as trackable drug nanocarriers for cancer treatment. MENs are heterostructures composed of a magnetostrictive core encased within a piezoelectric shell. Magnetostriction is a reversible property of ferromagnetic materials (e.g., cobalt ferrite), which causes them to expand or contract in response to a magnetic field. Piezoelectricity is the reversible appearance of a positive charge on one face and a negative charge on the opposite face (i.e., a voltage) of certain solid materials (e.g., fused silica, barium titanate) when they are subjected to mechanical stress. In a magneto-electric nanoparticle, application of a magnetic field will induce a change in the dimension of the magnetostrictive core which will transfer strain through the interface to the piezoelectric shell. Consequently, a charge polarization and change in zeta (2) potential is introduced on the shell surface through the piezoelectric process, which is the desired magneto-electric effect.

What is needed are compositions and methods for improving cancer treatment regimens to reduce systemic toxicity and positively impact the quality-of-life post-treatment. These compositions and methods would improve the anti-cancer efficacy and reduce the off-target toxicity for chemotherapeutic drugs, including anthracyclines such as Dox.

### SUMMARY

One embodiment described herein is a magnetoelectric nanoparticle composition for targeted and controlled drug release, the composition comprising a cobalt ferrite (CoFe₂O₄) magnetic nanoparticle core; a fused silica shell; and one or more therapeutic agents covalently conjugated to the fused silica shell. In one aspect, the one or more therapeutic agents comprise one or more chemotherapeutic drugs comprising doxorubicin or diphyllin. In another aspect, the composition further comprises a polyethylene glycol (PEG)-linked folate or folic acid molecule covalently conjugated to the fused silica shell. In another aspect, the cobalt ferrite magnetic nanoparticle core with the fused silica shell has a diameter of about 1 nm to about 18 nm. In another aspect, the cobalt ferrite magnetic nanoparticle core with the fused silica shell has a diameter of about 4 nm to about 8 nm. In another aspect, the one or more therapeutic agents are covalently conjugated to the fused silica shell through silanization with a succinic acid anhydride moiety on the fused silica shell.

Another embodiment described herein is a system for targeted and controlled drug release, the system comprising a magnetoelectric nanoparticle composition comprising a cobalt ferrite (CoFe₂O₄) magnetic nanoparticle core; a fused silica shell; and one or more therapeutic agents covalently conjugated to the fused silica shell; and an alternating current electromagnetic field source. In one aspect, the alternating current electromagnetic field source comprises an electromagnet coupled to a sinusoidal alternating current generator. In another aspect, the alternating current electromagnetic field source generates a magnetic field strength of about 20 Gauss to about 60 Gauss. In another aspect, the alternating current electromagnetic field source generates a magnetic field strength of about 25 Gauss to about 50 Gauss.

Another embodiment described herein is a method for treating a subject having cancer or at risk of developing cancer, the method comprising administering to the subject a therapeutically effective amount of a magnetoelectric nanoparticle composition comprising a cobalt ferrite (CoFe₂O₄) magnetic nanoparticle core; a fused silica shell; and one or more therapeutic agents covalently conjugated to the fused silica shell; and applying an external alternating current electromagnetic field to the subject, thereby releasing the one or more therapeutic agents from the administered magnetoelectric nanoparticle composition. In one aspect, the one or more therapeutic agents comprise one or more chemotherapeutic drugs comprising doxorubicin or diphyllin. In another aspect, the external alternating current electromagnetic field comprises a magnetic field strength of about 20 Gauss to about 60 Gauss. In another aspect, the external alternating current electromagnetic field comprises a magnetic field strength of about 25 Gauss to about 50 Gauss. In another aspect, the external alternating current electromagnetic field is applied to the subject for a period of time of about 1 minute to about 48 hours. In another aspect, the external alternating current electromagnetic field is applied to the subject at a frequency of about 50 Hz to about 150 Hz. In another aspect, the one or more therapeutic agents are released from the magnetoelectric nanoparticle composition at a primary tumor site, a metastatic tumor site, or a combination thereof in the subject. In another aspect, the therapeutically effective amount of the magnetoelectric nanoparticle composition is about 5 mg/kg to about 100 mg/kg. In another aspect, the therapeutically effective amount of the magnetoelectric nanoparticle composition is administered using a dosing regimen comprising a single dose or a plurality of doses.

Another embodiment described herein is a method of making a magnetoelectric nanoparticle composition for targeted and controlled release of doxorubicin, the method comprising mixing a magnetoelectric nanoparticle comprising a cobalt ferrite (CoFe₂O₄) magnetic nanoparticle core and a fused silica shell with (3-Triethoxysilyl) propylsuccinic anhydride to create a silanized magnetoelectric nanoparticle; and reacting the silanized magnetoelectric nanoparticle with doxorubicin hydrochloride to create the magnetoelectric nanoparticle composition. In one aspect, the (3-Triethoxysilyl) propylsuccinic anhydride is dispersed in a mixture of ethanol and water. In another aspect, reacting the silanized magnetoelectric nanoparticle with doxorubicin hydrochloride comprises incubating at a temperature of about 4° C. for a period of time of about 24 hours. In another aspect, the method further comprises reacting the silanized magnetoelectric nanoparticle with a polyethylene glycol (PEG)-linked folate or folic acid molecule in dimethyl formamide to covalently conjugate the PEG-linked folate or folic acid molecule to the silanized magnetoelectric nanoparticle. In another aspect, reacting the silanized magnetoelectric nanoparticle with the PEG-linked folate or folic acid molecule comprises a first incubation at room temperature for about 3 hours, and a second incubation at about 4° C. for about 24 hours.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-C show transmission electron microscopy (TEM) images showing CoFe₂O₄ nanoparticle cores (FIG. 1A), magnetoelectric silica nanoparticles (MagSiNs) (FIG. 1B), and magnetoelectric nanoparticles (MENs) (FIG. 1C). The scale bars for FIG. 1A are 20 nm (left) and 5 nm (right); the scale bars for FIG. 1B are 5 nm (left) and 5 nm (right); and the scale bars for FIG. 1C are 20 nm (left) and 20 nm (right). The crystal lattice of CoFe₂O₄ nanoparticles was clearly visible in the higher magnifications. The lattice spacing on the CoFe₂O₄ nanoparticle cores was 0.865±0.038 nm. FIG. 1D shows that from the TEM images, the MagSiNs diameter was 6.71±2.48 nm (top) and the MENs average diameter was 37.1±13.9 nm (bottom). The silica-shell thickness was 1.51±0.94 nm. The Barium Titanate shell thickness and shape was highly variable. FIG. 1E shows energy dispersive X-ray spectrum (EDXS) of MagSiNs and MENs that confirmed their distinct elemental composition differences in their shells. Distinct energy peaks for Fe (k_{α}: 6.398 keV), Co (k_{α}: 6.924 keV), Si (k_{α}: 1.739 keV), and O (k_{α}: 0.525 keV), were detected and labelled on MagSiNs samples. Distinct energy peaks for Fe (k_{α}: 6.398 keV), Co (k_{α}: 6.924 keV), Ba (L_{α}: 4.465 keV), Ti (k_{α}: 4.508 keV) and O (k_{α}: 0.525 keV), were detected and labelled on MENs samples. The EDXS peak heights are consistent with the stoichiometry of Co:Fe=1:2.

FIG. 2A-F show dual-mode detection of MagSiNs. Vibrating sample magnetometry was used to measure magnetization as a function of magnetic field of CoFe₂O₄ nanoparticles (FIG. 2A) and MagSiNs (FIG. 2B) in a reversible magnetic field at 300 K. T₁ and T₂ averaged MRI scans with reference to the concentration of CoFe₂O₄ nanoparticles were taken. FIG. 2C shows that CoFe₂O₄ nanoparticles showed a T₂-weighted effect with higher negative contrast at higher concentrations. FIG. 2D shows that MagSiNs with the silica shell showed a T₂-weighted, negative contrast effect only until 3 mM. However, it still demonstrated the efficacy of MagSiNs as MRI contrast agents. FIG. 2E shows the ratio of transverse/longitudinal relaxivity (r₂/r₁) for iron-oxide NPs and commercially available T₂-contrast agent RESOVIST™ when compared to the CoFe₂O₄ nanoparticles and MagSiNs. MRI measurements were carried out in a 1T benchtop MRI. FIG. 2F shows fluorophores embedded in the silica shell of MagSiNs to impart green fluorescence from FITC (top) or red fluorescence from RITC (bottom).

FIG. 3A-C show the assessment of the cytocompatibility and biocompatibility of MagSiNs to determine the usefulness of MagSiNs as drug nanocarriers. FIG. 3A shows the cytocompatibility of 0.116 μg of MagSiNs assessed using the blood-vessel model cell line HUVECs. FIG. 3B shows that a live/dead assay indicated that viable cells in control cells groups (84±10.5%) and viable cells in MagSiNs-exposed test cell groups (71±8.9%) had similar cell viability at 48 h post-exposure. FIG. 3C shows biocompatibility assessed using four-time cohorts of BalbcJ mice (4 h, 8 h, 24 h, 48 h) and histology. There were three mice per cohort and the mice in each cohort were sacked at the pre-determined time after a 10 mg/kg MagSiNs injection. The brain, lungs, heart, liver, spleen, kidney, blood, and fecal pellets from the intestines were extracted and fixed in 4% buffered paraformaldehyde. Histology was scored for inflammation by a board-certified pathologist. There was no inflammation in any of the cohorts. The MagSiNs were cytocompatible and biocompatible.

FIG. 4A-B show the assessment of the biodistribution of MagSiNs to determine the usefulness of MagSiNs as drug nanocarriers. FIG. 4A shows representative ex vivo T₂-weighted, negative contrast, MRI scans of the mouse organs from the different time cohorts using the Bruker 1T benchtop MRI. The T₂-weighted images were used to quantify the integrated intensity of the mouse organs from each time cohort. DI water and 10 mg/mL MagSiNs were used as control samples. FIG. 4B shows that the ratio of the T₂-weighted, integrated intensity of the signal from control mouse organs to that of the T₂-weighted, integrated intensity of the signal from MagSiNs injected mouse organs was used to determine biodistribution kinetics over 48 h. There was no non-specific accumulation of the MagSiNs in any organs. The MagSiNs exhibited T₂-MRI contrast and possessed favorable biodistribution, which made them suitable as drug nanocarriers. The results of FIG. 4B indicate good bio-clearance of MagSiNs from organs 48 hr post-injection.

FIG. 5A shows a schematic illustration of the conjugation of doxorubicin to succinic acid anhydride group on MagSiNs through the formation of an amide bond between the amine group of the doxorubicin hydrochloride (Dox·HCl) and the acid anhydride group on the MagSiNs. FIG. 5B shows a schematic illustration of the experimental workflow of the addition of free Dox·HCl to normal cells and cancer cells. The free Dox·HCl enters all cells, killing both normal and cancer cells alike. FIG. 5C shows a schematic illustration of the experimental workflow of the addition of silanized-Dox·HCl conjugated to magneto-electric silica nanoparticles (MagSiNs) to normal cells and cancer cells. The silanized-Dox·HCl conjugated to MagSiNs was incubated with the cells while exposed to a permanent magnet, followed by drug-release in an electromagnetic field. There was no cytotoxicity of the Dox-MagSiNs until the Dox·HCl was released from the Dox-MagSiNs by means of the externally applied electromagnetic field (30-50 Gauss). The illustrations of FIG. 5B-C are not drawn to scale.

FIG. 6A-D show total cells (green channel) versus dead cells (red channel) for four cell lines in chamber slides exposed to Doxorubicin (Dox·HCl) released from Dox-MagSiNs compared to appropriate control groups. All the treated cells and controls were exposed to a permanent magnet for 24 h, and then an alternating current magnetic field (AC magnetic field) of the same strength for 10 h. The live/dead assay was performed 48 h after initial drug exposure. FIG. 6A shows that for normal, control HUVECs, 100% cell death was observed after exposure to 500 nM Dox·HCl in its free form or after release from MagSiNs indicating Dox·HCl activity was not lost after immobilization on MagSiNs. FIG. 6B shows that for metastatic ovarian cancer cells (A2780), significant (83%) cell death was observed after exposure to 500 nM Dox·HCl in its free form. 53% cell death was observed after 500 nM Dox·HCl was released from Dox-MagSiNs, which was still significant against control untreated cells. FIG. 6C shows that for metastatic prostate cancer cells (PC-3), 100% cell death was observed after exposure to 500 nM Dox·HCl in its free form. 47% cell death was observed after 500 nM Dox·HCl was released from MagSiNs. FIG. 6D shows that for metastatic triple-negative breast cancer cells (MDA-MB-231), 4% cell death was observed after exposure to 500 nM Dox·HCl in its free form. About 10% cell death was observed after 500 nM Dox·HCl was released from Dox-MagSiNs, indicating increased Dox·HCl anti-cancer efficacy after release from Dox-MagSiNs. MagSiNs themselves or 500 nM Dox·HCl on Dox-MagSiNs were not found to be toxic to HUVEC cells or any of the cancer cells tested. In the tables below the bar graphs, ‘+’ indicates the presence of the component, while ‘-’ indicates the absence of the component.

FIG. 7 shows the results of co-localization assays performed for MagSiNs in different cell lines. The silica shell of MagSiNs was volume-loaded with Rhodamine-B red fluorescent dye. Lysosomes were stained with lysoview-green. Nuclei were stained with DAPI. Co-localization was assessed in the presence and in the absence of a 27-35 Gauss, permanent magnetic field. HUVEC cells showed no dependence on the external magnetic field for the co-localization of the MagSiNs extra-cellularly or intra-cellularly. Pearson's coefficient for co-localization with and without the magnetic field were 0.87 and 0.74, respectively. A2780 cells had well-defined lysosomes in the absence of a magnetic field. In the presence of a magnetic field, the MagSiNs were clustered and seemed to be co-localized with lysosomes in the overlay. Pearson's coefficient for co-localization with and without the magnetic field were 0.89 and 0.92, respectively. PC-3 cells had well-defined lysosomes. MagSiNs were co-localized in the lysosomes in the absence of a magnetic field (Pearson's co-localization coefficient=0.95). In the presence of a magnetic field, the majority of MagSiNs were not co-localized with the lysosomes even though a smaller population of well-delineated lysosomes remained. The Pearson's co-localization coefficient decreased to 0.86. MDA-MB-231 cells were densely packed with lysosomes. In the absence of a magnetic field, the MagSiNs were barely co-localized with the cells (Pearson's co-localization coefficient=0.77). In the presence of a magnetic field, the MagSiNs were heavily co-localized with the cells and especially with the lysosomes in the overlay, indicating efficient sequestration of the MagSiNs in the lysosomes (Pearson's co-localization coefficient=0.87).

FIG. 8 shows magnetoelectric nanoparticles (MENs) synthesized according to existing protocols in peer-reviewed literature (Rodzinski et al., Targeted and controlled anticancer drug delivery and release with magnetoelectric nanoparticles, Sci Rep, 6:20867 (2016)). The MENs have a cobalt ferrite core and a barium titanate piezoelectric shell with net diameters larger than 40 nm. It was difficult to control the thickness of the barium titanate shell consistently. Energy dispersive X-ray spectrum (EDXS) was used to confirm the elemental composition of the MENs.

FIG. 9 shows a fluorescence calibration curve used to estimate FITC release from MagSiNs. The excitation (Ex) wavelength is 494 nm; the emission (Em) wavelength is 518 nm; the Ex slit is 2.5 nm; and the Em slit is 2.5 nm.

FIG. 10A-D show illustrations of different modes of drug loading and release. FIG. 10A shows the use of CLICK chemistry to link the drug molecule covalently to the MagSiNs to investigate whether the drug was still therapeutically active while conjugated to the MagSiNs surface (i.e., no induced drug release). FIG. 10B shows the use of acid-labile ester linkers to release the drug payload from MagSiNs (i.e., bio-targeted pH sensitive drug release). An additional antibody for increased targeting to cancer cells may also be conjugated to the MagSiNs.

FIG. 10C shows the use of acid-labile ester linkers to release the drug payload from MagSiNs where magnetic fields were used for targeting MagSiNs to target cells (i.e., magnetically-targeted pH sensitive drug release). FIG. 10D shows electrostatic loading of drugs on MagSiNs followed by electromagnetic release of the drug payload (i.e., on demand drug release). FIG. 10E shows a graph demonstrating the high stability of covalently bound FITC to the MagSiNs surface at acidic pH 4.75 over 5 days at 37° C. and 5% CO₂. FIG. 10F shows a graph of the release rate of FITC bound to MagSiNs using acid-labile ester linkers as a function of the length of the carbon spacer (2-carbon or 4-carbon) linking the FITC to the ester functional group at acidic pH 4.75 over 4 days at 37° C. and 5% CO₂.

FIG. 11A-C show transwell migration assay results of MDA-MB-231 breast cancer cells (FIG. 11A), A2780 ovarian cancer cells (FIG. 11B), and PC-3 prostate cancer cells (FIG. 11C) administered MagSiNs conjugated to hydrophilic doxorubicin or hydrophobic diphyllin. Diphyllin released from the MagSiNs was found to inhibit transwell migration of all cancer cells tested.

FIG. 12A-C show live/dead cell assay results of PC-3 prostate cancer cells (FIG. 12A), A2780 ovarian cancer cells (FIG. 12B), and MDA-MB-231 triple negative breast cancer cells (FIG. 12C) administered MagSiNs conjugated to hydrophilic doxorubicin, hydrophobic diphyllin, or a combination of both chemotherapeutic drug molecules.

FIG. 13A-B show live/dead cell assay results of PC-3 prostate cancer cells (FIG. 13A) and A2780 ovarian cancer cells (FIG. 13B) administered folate conjugated MagSiNs conjugated to hydrophilic doxorubicin, hydrophobic diphyllin, or a combination of both chemotherapeutic drug molecules. FIG. 13C shows live/dead cell assay results of HUVEC cells and MDA-MB-231 breast cancer cells administered folate conjugated MagSiNs conjugated to hydrophilic doxorubicin, hydrophobic diphyllin, or a combination of both chemotherapeutic drug molecules. FIG. 13D shows live/dead cell assay results of MDA-MB-231 breast cancer cells administered folate conjugated MagSiNs conjugated to a combined formulation of doxorubicin and diphyllin in the presence or absence of an external magnetic field.

FIG. 14A-B show in vivo MagSiN localization to mice tumors. MDA-MB-231 cells expressing fluorescent RFP (MDA-231-RFP) were injected subcutaneously into Foxn1^{nu/nu} female mice and allowed to form tumors for 6 weeks. MagSiNs labeled with Dylight750 were then injected into the mice, and the mice were either not exposed to a magnetic field (FIG. 14A) or exposed to a 30-40 Gauss whole body permanent magnetic field (FIG. 14B) for 24 hr. Whole body imaging was performed to detect RFP from the tumor cells, and near-IR imaging was performed to detect MagSiN localization. The MagSiNs were found to localize to the primary tumor site and to metastatic sites derived from the MDA-MB-231 cancer cells in the mice exposed to the magnetic field. FIG. 14C-F show representative immunohistochemistry (IHC) images of the MDA-MB-231 cells in the primary tumor site of mice (FIG. 14C) and in other organs by staining for Cytokeratin-8, indicating brain metastasis (FIG. 14D), liver metastasis (FIG. 14E), and lung metastasis (FIG. 14F) in the mice. The brown IHC color indicates positive Cytokeratin-8 staining, indicating the presence of the MDA-MB-231 cells.

FIG. 15 shows representative IHC histology images from tumor tissue and different mouse organs for inflammation scoring following treatment with MagSiNs. Dox and a magnetic field.

FIG. 16 shows schematic illustrations of folic acid-conjugated MagSiNs loaded with chemotherapeutics.

FIG. 17 shows live/dead cell assay results of HUVEC cells and MDA-MB-231 breast cancer cells administered folic acid-conjugated MagSiNs loaded with doxorubicin and/or diphyllin chemotherapeutic drugs.

FIG. 18 shows Lysotracker assay results of HUVEC cells and MDA-MB-231 breast cancer cells administered folic acid-conjugated MagSiNs loaded with drug with or without a magnetic field. The ratio of the fluorescence intensity of MagSiN nanoparticles (rhodamine fluorescence) and lysosomes fluorescence intensity per cell was plotted for HUVEC cells and MDA-MB-231 cells. The magnetic field was 30-50 Gauss.

FIG. 19 shows live/dead cell assay results of HUVEC cells administered non-folate-conjugated drug-MagSiNs and exposed to an alternating magnetic field to trigger drug release.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

One embodiment described herein is a magnetoelectric nanoparticle composition for targeted and controlled drug release, the composition comprising a cobalt ferrite (CoFe₂O₄) magnetic nanoparticle core; a fused silica shell; and one or more therapeutic agents covalently conjugated to the fused silica shell. In one aspect, the one or more therapeutic agents comprise one or more chemotherapeutic drugs comprising doxorubicin or diphyllin. In another aspect, the composition further comprises a polyethylene glycol (PEG)-linked folate or folic acid molecule covalently conjugated to the fused silica shell. In another aspect, the cobalt ferrite magnetic nanoparticle core with the fused silica shell has a diameter of about 1 nm to about 18 nm. In another aspect, the cobalt ferrite magnetic nanoparticle core with the fused silica shell has a diameter of about 4 nm to about 8 nm. In another aspect, the one or more therapeutic agents are covalently conjugated to the fused silica shell through silanization with a succinic acid anhydride moiety on the fused silica shell.

Another embodiment described herein is a system for targeted and controlled drug release, the system comprising a magnetoelectric nanoparticle composition comprising a cobalt ferrite (CoFe₂O₄) magnetic nanoparticle core; a fused silica shell; and one or more therapeutic agents covalently conjugated to the fused silica shell; and an alternating current electromagnetic field source. In one aspect, the alternating current electromagnetic field source comprises an electromagnet coupled to a sinusoidal alternating current generator. In another aspect, the alternating current electromagnetic field source generates a magnetic field strength of about 10 Gauss to about 300 Gauss. In another aspect, the alternating current electromagnetic field source generates a magnetic field strength of about 20 Gauss to about 100 Gauss. In another aspect, the alternating current electromagnetic field source generates a magnetic field strength of about 20 Gauss to about 60 Gauss. In another aspect, the alternating current electromagnetic field source generates a magnetic field strength of about 25 Gauss to about 50 Gauss.

Another embodiment described herein is a method for treating a subject having cancer or at risk of developing cancer, the method comprising administering to the subject a therapeutically effective amount of a magnetoelectric nanoparticle composition comprising a cobalt ferrite (CoFe₂O₄) magnetic nanoparticle core; a fused silica shell; and one or more therapeutic agents covalently conjugated to the fused silica shell; and applying an external alternating current electromagnetic field to the subject, thereby releasing the one or more therapeutic agents from the administered magnetoelectric nanoparticle composition. In one aspect, the one or more therapeutic agents comprise one or more chemotherapeutic drugs comprising doxorubicin or diphyllin. In another aspect, the external alternating current electromagnetic field comprises a magnetic field strength of about 10 Gauss to about 300 Gauss. In another aspect, the external alternating current electromagnetic field comprises a magnetic field strength of about 20 Gauss to about 100 Gauss. In another aspect, the external alternating current electromagnetic field comprises a magnetic field strength of about 20 Gauss to about 60 Gauss. In another aspect, the external alternating current electromagnetic field comprises a magnetic field strength of about 25 Gauss to about 50 Gauss. In another aspect, the external alternating current electromagnetic field is applied to the subject for a period of time of about 1 minute to about 48 hours. In another aspect, the external alternating current electromagnetic field is applied to the subject at a frequency of about 5 Hz to about 500 Hz. In another aspect, the external alternating current electromagnetic field is applied to the subject at a frequency of about 50 Hz to about 150 Hz. In another aspect, the one or more therapeutic agents are released from the magnetoelectric nanoparticle composition at a primary tumor site, a metastatic tumor site, or a combination thereof in the subject. In another aspect, the therapeutically effective amount of the magnetoelectric nanoparticle composition is about 1 mg/kg to about 500 mg/kg. In another aspect, the therapeutically effective amount of the magnetoelectric nanoparticle composition is about 5 mg/kg to about 100 mg/kg. In another aspect, the therapeutically effective amount of the magnetoelectric nanoparticle composition is administered using a dosing regimen comprising a single dose or a plurality of doses.

In some embodiments of the present invention, a subject is administered a therapeutically effective amount of a magnetoelectric nanoparticle composition using a specific dosing regimen. In one aspect, the dosing regimen comprises a single dose of the therapeutically effective amount of the magnetoelectric nanoparticle composition administered at a single point in time. In another aspect, the dosing regimen comprises a plurality of doses of the therapeutically effective amount of the magnetoelectric nanoparticle composition administered over a period of time. For example, in various nonlimiting embodiments, a magnetoelectric nanoparticle composition as described herein may be administered to a subject once a day (SID/QD), twice a day (BID), three times a day (TID), four times a day (QID), or more, so as to administer a therapeutically effective amount of the magnetoelectric nanoparticle composition to the subject, where the therapeutically effective amount is any one or more of the doses described herein. In some embodiments, a pharmaceutical composition as described herein is administered to a subject 1-3 times per day, 1-7 times per week, 1-9 times per month, 1-12 times per year, or more. In other embodiments, a magnetoelectric nanoparticle composition is administered for about 1-10 days, 10-20 days, 20-30 days, 30-40 days, 40-50 days, 50-60 days, 60-70 days, 70-80 days, 80-90 days, 90-100 days, 1-6 months, 6-12 months, 1-5 years, or more. In various embodiments, a pharmaceutical composition as described herein is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, 900-1000 mg/kg, or a combination thereof.

The actual dosing regimen can depend upon many factors, including but not limited to the judgment of a trained physician, the overall condition of the subject, the age of the subject, and the specific type and stage of cancer. The actual dosage can also depend on the determined experimental effectiveness of the specific magnetoelectric nanoparticle composition that is administered. For example, the dosage may be determined based on in vitro responsiveness of relevant cultured cells, or in vivo responses observed in appropriate animal models or human studies.

Another embodiment described herein is a method of making a magnetoelectric nanoparticle composition for targeted and controlled release of doxorubicin, the method comprising mixing a magnetoelectric nanoparticle comprising a cobalt ferrite (CoFe₂O₄) magnetic nanoparticle core and a fused silica shell with (3-Triethoxysilyl) propylsuccinic anhydride to create a silanized magnetoelectric nanoparticle; and reacting the silanized magnetoelectric nanoparticle with doxorubicin hydrochloride to create the magnetoelectric nanoparticle composition. In one aspect, the (3-Triethoxysilyl) propylsuccinic anhydride is dispersed in a mixture of ethanol and water. In another aspect, reacting the silanized magnetoelectric nanoparticle with doxorubicin hydrochloride comprises incubating at a temperature of about 4° C. for a period of time of about 24 hours. In another aspect, the method further comprises reacting the silanized magnetoelectric nanoparticle with a polyethylene glycol (PEG)-linked folate or folic acid molecule in dimethyl formamide to covalently conjugate the PEG-linked folate or folic acid molecule to the silanized magnetoelectric nanoparticle. In another aspect, reacting the silanized magnetoelectric nanoparticle with the PEG-linked folate or folic acid molecule comprises a first incubation at room temperature (i.e., about 20° C. to about 25° C.) for about 1 hour to about 5 hours (e.g., about 3 hours), and a second incubation at about 4° C. for about 24 hours.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A magnetoelectric nanoparticle composition for targeted and controlled drug release, the composition comprising:
   a cobalt ferrite (CoFe₂O₄) magnetic nanoparticle core;
   a fused silica shell; and
   one or more therapeutic agents covalently conjugated to the fused silica shell.
Clause 2. The composition of clause 1, wherein the one or more therapeutic agents comprise one or more chemotherapeutic drugs comprising doxorubicin or diphyllin.
Clause 3. The composition of clause 1 or 2, further comprising a polyethylene glycol (PEG)-linked folate or folic acid molecule covalently conjugated to the fused silica shell.
Clause 4. The composition of any one of clauses 1-3, wherein the cobalt ferrite magnetic nanoparticle core with the fused silica shell has a diameter of about 1 nm to about 18 nm.
Clause 5. The composition of any one of clauses 1-4, wherein the cobalt ferrite magnetic nanoparticle core with the fused silica shell has a diameter of about 4 nm to about 8 nm.
Clause 6. The composition of any one of clauses 1-5, wherein the one or more therapeutic agents are covalently conjugated to the fused silica shell through silanization with a succinic acid anhydride moiety on the fused silica shell.
Clause 7. A system for targeted and controlled drug release, the system comprising:
   a magnetoelectric nanoparticle composition comprising:
      a cobalt ferrite (CoFe₂O₄) magnetic nanoparticle core;
      a fused silica shell; and
      one or more therapeutic agents covalently conjugated to the fused silica shell; and
   an alternating current electromagnetic field source.
Clause 8. The system of clause 7, wherein the alternating current electromagnetic field source comprises an electromagnet coupled to a sinusoidal alternating current generator.
Clause 9. The system of clause 7 or 8, wherein the alternating current electromagnetic field source generates a magnetic field strength of about 20 Gauss to about 60 Gauss.
Clause 10. The system of any one of clauses 7-9, wherein the alternating current electromagnetic field source generates a magnetic field strength of about 25 Gauss to about 50 Gauss.
Clause 11. A method for treating a subject having cancer or at risk of developing cancer, the method comprising:
   administering to the subject a therapeutically effective amount of a magnetoelectric nanoparticle composition comprising:
      a cobalt ferrite (CoFe₂O₄) magnetic nanoparticle core;
      a fused silica shell; and
      one or more therapeutic agents covalently conjugated to the fused silica shell; and
   applying an external alternating current electromagnetic field to the subject, thereby releasing the one or more therapeutic agents from the administered magnetoelectric nanoparticle composition.
Clause 12. The method of clause 11, wherein the one or more therapeutic agents comprise one or more chemotherapeutic drugs comprising doxorubicin or diphyllin.
Clause 13. The method of clause 11 or 12, wherein the external alternating current electromagnetic field comprises a magnetic field strength of about 20 Gauss to about 60 Gauss.
Clause 14. The method of any one of clauses 11-13, wherein the external alternating current electromagnetic field comprises a magnetic field strength of about 25 Gauss to about 50 Gauss.
Clause 15. The method of any one of clauses 11-14, wherein the external alternating current electromagnetic field is applied to the subject for a period of time of about 1 minute to about 48 hours.
Clause 16. The method of any one of clauses 11-15, wherein the external alternating current electromagnetic field is applied to the subject at a frequency of about 50 Hz to about 150 Hz.
Clause 17. The method of any one of clauses 11-16, wherein the one or more therapeutic agents are released from the magnetoelectric nanoparticle composition at a primary tumor site, a metastatic tumor site, or a combination thereof in the subject.
Clause 18. The method of any one of clauses 11-17, wherein the therapeutically effective amount of the magnetoelectric nanoparticle composition is about 5 mg/kg to about 100 mg/kg.
Clause 19. The method of any one of clauses 11-18, wherein the therapeutically effective amount of the magnetoelectric nanoparticle composition is administered using a dosing regimen comprising a single dose or a plurality of doses.
Clause 20. A method of making a magnetoelectric nanoparticle composition for targeted and controlled release of doxorubicin, the method comprising:
   mixing a magnetoelectric nanoparticle comprising a cobalt ferrite (CoFe₂O₄) magnetic nanoparticle core and a fused silica shell with (3-Triethoxysilyl) propylsuccinic anhydride to create a silanized magnetoelectric nanoparticle; and
   reacting the silanized magnetoelectric nanoparticle with doxorubicin hydrochloride to create the magnetoelectric nanoparticle composition.
Clause 21. The method of clause 20, wherein the (3-Triethoxysilyl) propylsuccinic anhydride is dispersed in a mixture of ethanol and water.
Clause 22. The method of clause 20 or 21, wherein reacting the silanized magnetoelectric nanoparticle with doxorubicin hydrochloride comprises incubating at a temperature of about 4° C. for a period of time of about 24 hours.
Clause 23. The method of any one of clauses 20-22, further comprising reacting the silanized magnetoelectric nanoparticle with a polyethylene glycol (PEG)-linked folate or folic acid molecule in dimethyl formamide to covalently conjugate the PEG-linked folate or folic acid molecule to the silanized magnetoelectric nanoparticle.
Clause 24. The method of any one of clauses 20-23, wherein reacting the silanized magnetoelectric nanoparticle with the PEG-linked folate or folic acid molecule comprises a first incubation at room temperature for about 3 hours, and a second incubation at about 4° C. for about 24 hours.

### EXAMPLES

### Example 1

### Materials and Methods

Cobalt nitrate hexahydrate, iron nitrate nonahydrate, polyvinylpyrrolidone (40 kDA), sodium borohydride, de-ionized water (DI H2O), Tetraethyl orthosilicate (TEOS), 20% w/v ammonium hydroxide (NH₄OH), 30% w/v ammonium hydroxide (NH₄OH), 200 proof ethanol, fluorescein isothiocyanate (FITC), rhodamine isothiocyanate (RITC), amino propyl triethoxy silane (APTES), 3-triethoxysilylpropylsuccinic anhydride (SSA), doxorubicin hydrochloride (Dox·HCl), 4% buffered paraformaldehyde, butanolamine, and ethanolamine. Dulbecco's Modified Eagle's Medium (DMEM), RPMI-1640, fetal bovine serum (FBS), and Trypsin-EDTA were purchased from Sigma-Aldrich (St. Louis, MO, USA). Complete endothelial cell growth medium was from R&D systems. MDA-MB-231, PC-3, A2780, and HUVEC were from ATCC. Lysoview-green, and Calcein-AM/ethidium homodimer III LIVE/DEAD assay were purchased from Thermo Fisher Scientific (Waltham, MA, USA).

### Nanoparticle Synthesis

### Synthesis of MagSiNs Core

The MagSiNs core (3-6 nm) was composed of CoFe₂O₄, and it was synthesized using the hydrothermal method. In beaker 1, 0.58 g of cobalt nitrate hexahydrate and 1.6 g iron nitrate nonahydrate were dissolved in 150 mL of deionized (DI) water. The contents of the beaker were stirred at 1000 RPM at 70° C. In beaker 2, 2 g of polyvinylpyrrolidone (40,000 molecular weight) and 9 g of sodium borohydride were dissolved in 50 mL of DI water. The beaker 2 solution was then added dropwise into beaker 1, at 0.55 mL/minute. Once this addition had been completed, the temperature of the hotplate was increased to 90° C., the stirring was decreased to 300 RPM, and the solution was left to sit while the water evaporated from the solution until the mass was very sticky and tar-like. Next, the stir bar was removed from the container, and 150 mL of DI water was added to the beaker, and the nanoparticle-core mass was sonicated in the ultrasound bath for at least 15 minutes. The CoFe₂O₄ nanoparticles were then magnetically separated from the supernatant. This process of rinsing the MagSiNs cores with DI water was repeated 3 more times followed by 3 rinses in ethanol. The cores were dried in an oven at 60° C. and stored as a powder at room-temperature until addition of the silica shell.

### Synthesis of Silica Shell on MagSiNs Cores

The materials required for the synthesis of the silica shell on the Mag-E-Si—N cores were 200 proof ethanol, tetraethyl orthosilicate (TEOS), 20% w/v ammonium hydroxide (NH₄OH), a sonic dismembrator, a centrifuge, an overhead non-magnetic stirrer, and a 400 ml beaker. 57 mg of the nanoparticle-cores was weighed out and added to a 50 mL centrifuge tube. 10-20 mL of 200 proof ethanol were added, and the tube was then placed in the sonic dismembrator and sonicated for 60 seconds at 40% amplitude (pulse on for 1 second, pulse off for 0.5 seconds). The core solution was then transferred to the 400 ml beaker, and additional 200 proof ethanol was added to make the total volume of ethanol 99 mL. Next, 1.05 mL of TEOS was added, and the solution was sonicated again for 20 seconds at 40% amplitude. The beaker of the solution was then stirred with the overhead stirrer. While spinning, 3 mL of 30% w/v NH₄OH was added. The container was sealed, and the cores were left to spin for approximately 48 h. After 48 h, the core-shell MagSiNs were rinsed three times in ethanol by centrifugation at 9000 RPM for 30 minutes each and finally stored at room temperature (i.e., about 20° C. to about 25° C.) as a dried pellet. The MagSiNS were resuspended in between rinses by using sonication at 40-50% power settings.

### Synthesis of Fluorescent MagSiNs

Green fluorescent molecules (FITC) or red fluorescen molecules (RITC) incorporated MagSiNs were synthesized in a similar manner with minor modifications. The particles in 30 mL anhydrous ethanol were dried and 22.6 mg was weighed out and resuspended in 40 mL of 200 proof ethanol in a 45 mL centrifuge tube. The solution was sonicated at 60% amplitude for 30 sec. After, 340 μL of tetraethyl orthosilicate APTES (TEOS) was added. The solution was transferred to a 100 mL flask and was then covered with aluminum foil before 92 μL of the RITC fluorophore was added. The beaker was taken to the fume hood and placed under an overhead stirrer on low spin speed. Finally, 875 μL of 30% ammonium hydroxide was added before parafilm was placed on the top of the flask to reduce ethanol evaporation and more aluminum foil was added to reduce light exposure to the fluorophore. The solution spun for 24 h before being washed three times in 35 mL of 200 proof ethanol for 15 minutes in the centrifuge at 9000 RPM. After the final wash, the cores were resuspended in 30 mL ethanol and stored. The same process was conducted for the addition of the 92 μL FITC fluorophore to 22.4 mg of cores.

### Nanoparticle Characterization

### Transmission Electron Microscope (TEM)

High-resolution transmission electron microscopy images (TEM) of silica capped cobalt-ferrite nanoparticles with a magnetic core and piezoelectric shell was carried out on a JEOL 2011 at 100 kV.

### Vibrating Sample Magnetometer (VSM)

The total magnetic moment of cobalt ferrite nanoparticles and MagSiNs at saturation magnetic field strength were measure using a vibrating sample magnetometer (VSM) for in-plane and out-of-plane measurements. The VSM was measured using a Microsense EV7 VSM.

### Magnetic Resonance Imaging (MRI)

The longitudinal relaxation time (r₁) and transverse relaxation time (r₂) were determined for cobalt ferrite nanoparticles, and MagSiNs using a 1T Bruker Benchtop icon magnetic-resonance imaging instrument (MRI) in order to assess their suitability as MRI image contrast agents. Magnetic resonance imaging was performed with a Bruker Icon 1T MRI scanner running Paravision 6.0.1 for preclinical MRI research. CoFe₂O₄ and MagSiNs were diluted to 1 mM, 3 mM, and 10 mM concentrations in deionized water. After a three-plane localizer scan, T₂ relaxation time was acquired with an MSME sequence protocol (T₂map-MSME). Echo time (TE) was varied from 18 to 198 with 18-ms increments with the following parameters: TR=2500 ms, matrix=192×192, FOV=35×35 mm, resolution=0.182×0.182 mm, bandwidth=15,000 Hz, slice thickness=1.250 mm, and total acquisition time=8 min. T₁-weighted MR images were acquired using a T₁ Rapid Imaging with Refocused Echoes (RARE) sequence (T₁_RARE) at various repetition times (TR) under the following parameters: TE=12.0 ms, TR=161.4, 400, 700, 1000, 1300, 1600 ms, matrix=128×128, FOV=30.0×30.0 mm, resolution=0.234×0.234, bandwidth=12,500 Hz, slice thickness=1 mm, and total acquisition time=33 min. T₂ relaxation time was measured after selecting a region-of-interest (ROI) from the generated T₂ maps. Signal Intensity (SI) was measured with ROIs from the generated T₁ images at various TRs. In Matlab (Mathworks), SI versus TR were plotted, and a two-parameter fit was performed to calculate Ti using the following equation:

$$Mz ( t ) = Mo ( 1 - {e}^{( - t / {T}_{1} )} ) .$$

Further image analysis was performed with ImageJ. The r₁ and r₂ values were calculated by determining the slope of 1/T₁ and 1/T₂ (s⁻¹) versus sample concentration (mM).

### Fluorescence Microscopy

Fluorescence of the cores after the addition of the fluorophores was analyzed via epi-fluorescent microscopy (Nikon eclipse 400 Melville, NY, USA) using standard green (fluorescein) and red (rhodamine) filter cubes.

### Zeta Potential Measurement in the Presence of Magnetic Field

The zeta potential of MagSiNs was measured to characterize the zeta potential with and without the influence of a magnetic field. The magnetic field was applied perpendicular to the electrical field of the electrodes. 1 mL of DI water and 20 μL of the MagSiNs was used as a solution. The refractive index of silica was used to calibrate the light scattering measurements. DLS and Zeta potential were measured at room temperature. Deionized water was the solvent. Each DLS or Zeta potential measurement file consisted of 3 runs. Each run was from an average of at least 15 measurement readings. The zeta potential was measured using a Malvern Panalytical Zetasizer Nano ZS/ZSE.

### Linking Drug-Proxy (FITC) to MagSiNs

MagSiNs Surface Functionalized with FITC (FITC-MagSiNs)

Aminopropyltriethoxysilane was reacted with FITC (APTES-FITC) in 1:1 mole ratio, using THF as a solvent under Nitrogen atmosphere. The reaction was allowed to proceed for 24 h under room temperature. The solution was then stored in −20° C. MagSiNs and APTES-FITC were mixed in 1:10 mole ratio in a 90/10 ethanol/water solution and allowed to stir at room temperature for 24 h. After 24 h, the silanized nanoparticles (MagSiNs-SSA) were magnetically separated from solution, rinsed in DI water twice, and resuspended in DI water. The amount of fluorophore on the -FITC-MagSiNs was quantified using fluorescence spectroscopy and comparison of fluorescence signal to a standard fluorescence calibration curve.

MagSiNs Surface Functionalized with FITC Through an Ethyl Ester Linker (MagSiNs-Ethyl-FITC)

MagSiNs were functionalized with acid anhydride group using silanization with 3-(triethoxysilyl) propylsuccinic anhydride (SSA) in 90/10 ethanol/water solution. Silanization was carried out for 24 h at room temperature. After 24 h, the silanized nanoparticles (MagSiNs-SSA) were magnetically separated from solution, rinsed in DI water twice, and resuspended in DI water. The acid anhydride groups on MagSiNs-SSA were reacted with 1000-fold mole excess of rhodamine tagged ethanolamine (ethanolamine-FITC) for 24 h at 4° C. After 24 h, the MagSiNs-SSA were magnetically separated from solution, rinsed in DI water twice, lyophilized and stored at −20° C. The amount of fluorophore on the MagSiNs-ethyl-FITC was quantified using fluorescence spectroscopy and comparison of fluorescence signal to a standard fluorescence calibration curve.

MagSiNs Surface Functionalized with FITC Through a Butyl Ester Linker (MagSiNs-Butyl-FITC)

MagSiNs were functionalized with FITC through a 4-carbon linker (butanolamine-FITC) using the same experimental workflow as that of MagSiNs surface functionalized with FITC through an ethyl ester linker. The amount of fluorophore on the MagSiNs-butyl-FITC was quantified using fluorescence spectroscopy and comparison of fluorescence signal to a standard fluorescence calibration curve.

Kinetics of Drug-Proxy (FITC) Release from MagSiNS for Different Payload Release Mechanisms ON-Demand FITC Release

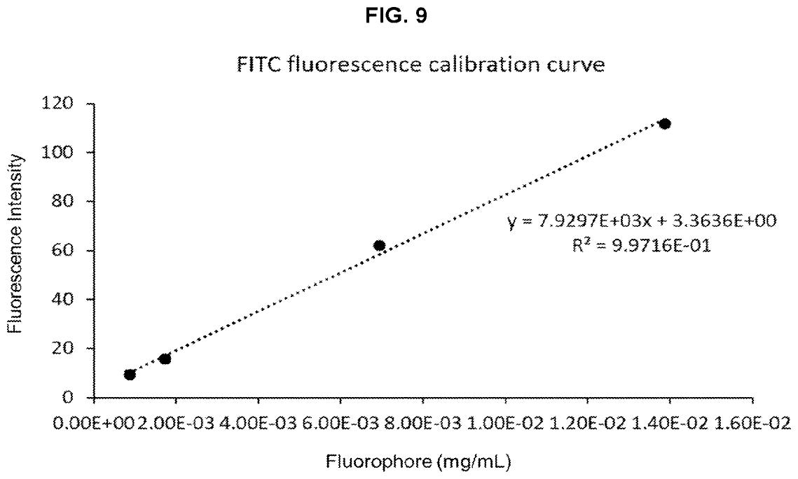

An alternating magnetic field of 100 Hz with a field strength in the range of 27-35 Gauss was applied to vials of MagSiNs-FITC in phosphate-buffered saline, in a 5% CO₂ cell incubator at 37° C. Vials were removed at 0.5 h, 1 h, 1.5 h, 3 h, 8 h, the nanoparticles were spun out, and the supernatants' fluorescence signals were measured. The amount of fluorophore in the supernatant was quantified by comparing the fluorescence intensity to a standard calibration curve (FIG. 9).

### Acid-Labile Ester Hydrolysis Dependent FITC Release

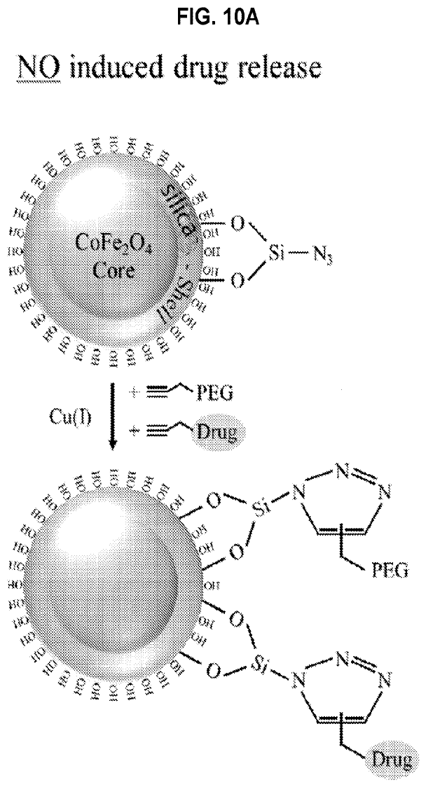

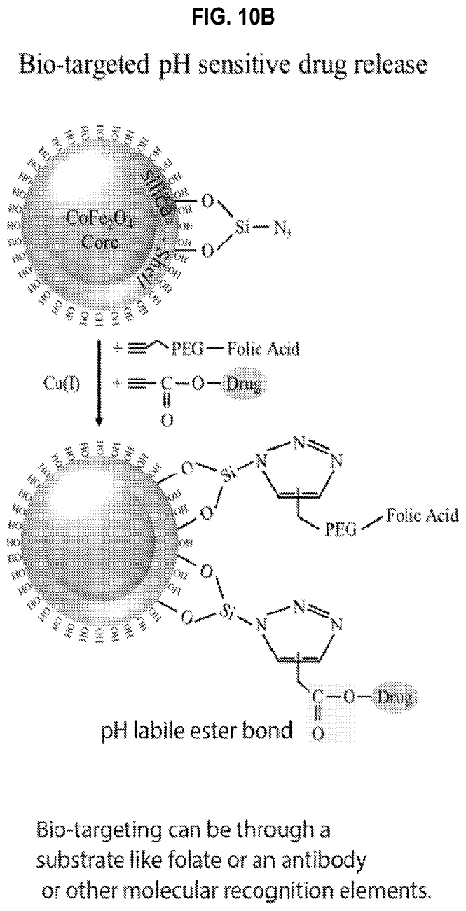

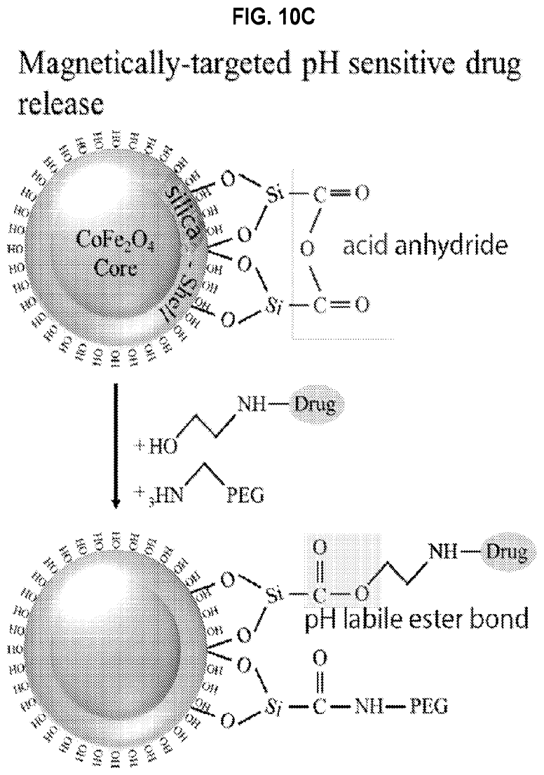

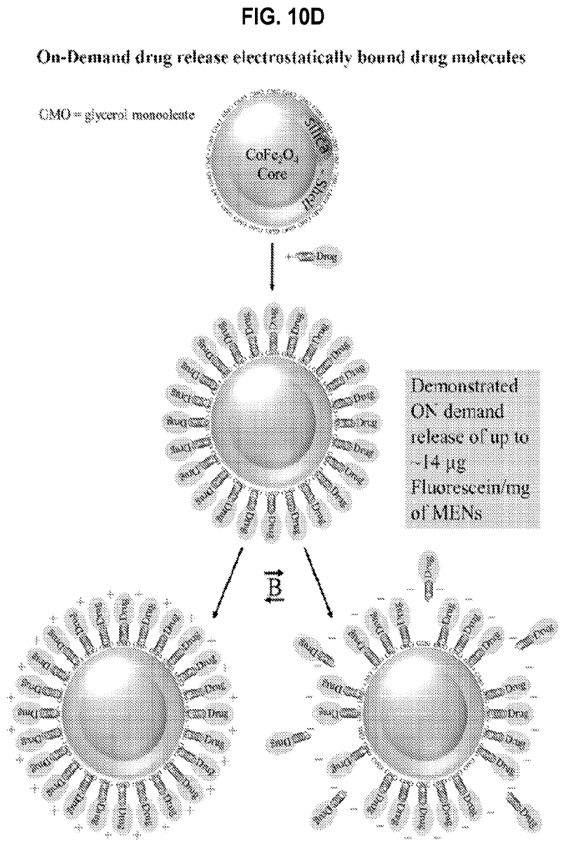

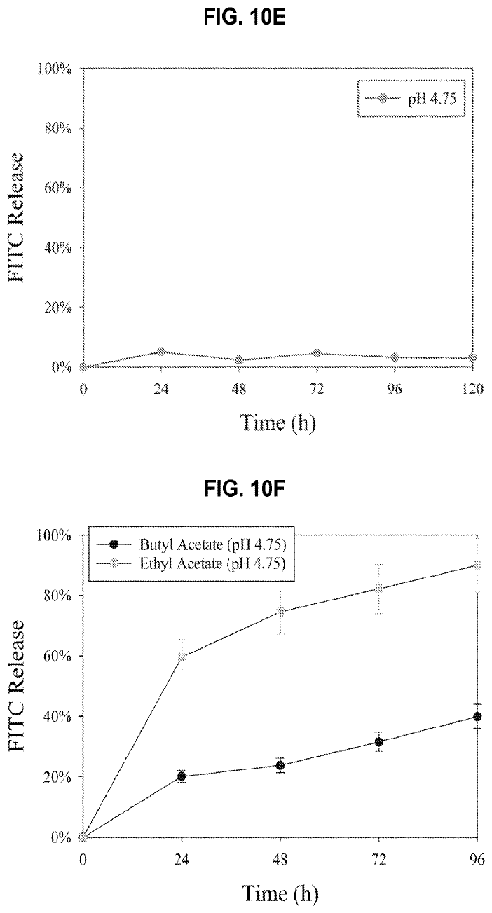

MagSiNs-ethyl-FITC and MagSiNs-butyl-FITC (FIG. 10) were suspended in vials of either phosphate-buffered saline at pH 7.2 or MES buffer at pH 4.75. The sample vials in pH 7.2 of 4.75 were placed in a 5% CO₂ cell incubator at 37° C. The vials were sampled at 24 h, 48 h, 72 h, and 96 h, the nanoparticles were spun out, and the supernatants' fluorescence signals were measured. The amount of fluorophore in the supernatant was quantified by comparing the fluorescence intensity to a standard calibration curve (FIG. 9).

### Linking Doxorubicin Hydrochloride (Dox·HCl) to MagSiNs (Dox-MagSiNs)

The MagSiNS were silanized with succinic acid. Typically, 100 μL of (3-Triethoxysilyl) propylsuccinic anhydride, 95% was dispersed in 5 mL of a 90/10 (v/v) mixture of 200 proof ethanol and deionized (DI) water and then added to 5 mg of MagSiNs. The mixture was allowed to stir overnight. The silanized MagSiNs were then magnetically separated from solution three times and rinsed in DI water. Zeta potential measurements using Malvern panalytical Zetasizer Nano ZS/ZSE were used to confirm the presence of succinic acid anhydride (SSA) on the surface of MagSiNs by monitoring the dramatic change in zeta potential between SSA functionalized MagSiNs (−16.13±0.76 mV) vs. non-functionalized MagSiNs (−6.8 mV).

Dox·HCl has an amine group on the cyclo-hexane group present in its structure. This amine group was reacted with the acid anhydride on MagSiNs-SSA in sterile DI water. The reaction was carried at 4° C. for 24 h, after which the Dox functionalized nanoparticles (Dox-MagSiNs) were magnetically separated from solution, rinsed in DI water twice, lyophilized, and stored at −20° C. Dox·HCl is red in color with a distinct UV-Vis spectrum in the visible range. The absorbance maximum of Dox·HCl is 480 nm. A calibration curve for known concentration of Dox·HCl was constructed using the absorbance max at 480 nm. After conjugating known mass of Dox·HCl to the SSA functionalized MagSiNs, the MagSiNs were spun out using centrifugation, and the supernatant was analyzed for mass of unbound Dox·HCl. From the unbound mass of Dox·HCl, the amount of Dox·HCl that was loaded on the NPs was determined. The amount of Dox·HCl loaded on the MagSiNs-Dox was determined using UV-Vis spectroscopy to determine absorbance at 480 nm and comparing that absorbance to a standard calibration curve for Dox·HCl.

### Linking Folate and Doxorubicin to MagSiNs (Folate Conjugated Dox-MagSiNs)

Similar to previous silanization, (v/v) 200 μL mixture of 3-azidopropyltriethoxysilane and (3-Triethoxysilyl) propylsuccinic anhydride were dispersed in 5 mL of a 90:10 (v/v) mixture of 200 proof ethanol and deionized (DI) water and then added to 5 mg of MagSiNs. The mixture was allowed to stir overnight. The silanized MagSiNs were then magnetically separated from solution three times and rinsed in DI water. The resulting MagSiNs had an azide functional group and an acid anhydride functional group (MagSiNs-SSA-Alkyne). Dox has an amine group on the cyclo-hexane group present in its structure. This amine group was reacted with the acid anhydride on MagSiNs-SSA-alkyne in sterile DI water. The reaction was carried at 4° C. for 24 hr, after which the Dox functionalized nanoparticles (Dox-MagSiNs) were magnetically separated from solution, rinsed in DI water twice, lyophilized and stored at −20° C. The amount of Dox·HCl loaded on the MagSiNs-Dox was determined using UV-Vis spectroscopy to determine absorbance at 480 nm and then comparing that absorbance to a standard calibration curve for Dox·HCl.

To link folate to the Dox-MagSiNs-alkyne, folate-PEG2k-alkyne (Nanocs) was used. Copper catalyzed CLICK chemistry was performed to link 1 mg of the folate-PEG2k-alkyne to the azide on 5 mg of the Dox-MagSiNs in dimethyl formamide under stirring at room temperature for 3 hr, followed by stirring in the refrigerator for 24 hr, after which the Dox and folate functionalized nanoparticles (Dox-MagSiNs) were magnetically separated from solution, rinsed in DI water twice, lyophilized, and stored at −20° C. Liquid chromatography coupled with mass spectrometry analysis of the supernatant after each linking step allowed for the determination of unbound Dox and unbound folate, from which the mass of Dox and the mass of folate conjugated to the MagSiNs were calculated.

### Magnetic Field Exposures

An electromagnet array was used for exposing cells to unidirectional magnetic field by passing a DC current, or an alternating magnetic field by passing an AC current through the electromagnet array. The electromagnet array consisted of 2×6 (12 in total) electromagnets with each row of electromagnets connected in series. A Hewlett-Packard S33120A waveform generator and a Krohn-Hite Model 7500 Wideband Power Amplifier 115/230 V 50-400 Hz were used to ensure that the electromagnets exhibited a magnetic field of between 23 Gauss to 40 Gauss. A DC current was applied to generate a unidirectional magnetic field. An AC current with a square waveform and 100 Hz frequency was used to generate an alternating magnetic field to trigger drug release.

### Cell Culture

### Cell Stock

For in vitro experimentation, a water bath, centrifuge, and a tissue culture hood were used, along with nutrient-rich media. Three cancer cell lines were cultured: MDA-MB-231 (breast cancer), A2780 (ovarian cancer), and PC-3 (prostate cancer). One control cell line, human umbilical vein endothelial cell (HUVEC), was also cultured. The cells were cultured in a 5% CO₂ incubator at 100% humidity. With regard to the nutrient-rich media, the type varied with each cell line. Dulbecco's Modified Eagle's Medium (DMEM) plus 10% FBS was used for MDA-MB-231. RPMI-1640 plus 10% FBS and 1% L-glutamine was used for A2780 and PC-3. Endothelial Cell Growth Medium (R&D systems) was used for the HUVEC cell line.

### Seeding of Experimental Chamber Slides

To seed the cells, 8-well chamber slides, glass cover slide, and a hemocytometer were needed. The cells were ideally at 70% to 80% confluence before seeding. 15,000-20,000 cells were seeded into each well of the chamber slides, along with 400 μL of complete cell growth media appropriate for each cell line.

### Cell Assays

### Cell Viability Assay

The cell viability assay consisted of seeding of cells into chamber slides, adding Dox (free or as Dox-MagSiNS), exposing slides to a permanent magnet for 24 h, exposing to an alternating current (AC) magnetic field for 10 h, and then performing a live/dead assay on the cells. The media in each well of the chamber slides was carefully aspirated, and the wells were rinsed twice with 400 μL of 1×PBS. The cells were incubated with Ethidium-homodimer Ill dye and Calcein-AM dye mix at manufacturer recommended concentrations (Life-technologies), and the slides were incubated for 30 minutes in the cell incubator, in the dark. After incubation, the wells were rinsed twice with 400 μL of 1×PBS. Lastly, 300 μL of 4% buffered parafomaldehyde was added to each well, and the slides were taken to be imaged with a NikonU inverted fluorescent microscope with a 10× lens using a standard FITC/RITC green and red fluorescent filter cubes.

### Intra-Cellular Co-Localization Assay

HUVEC, A2780, MDA-MB-231, and PC-3 cells were plated onto sterile coverslips and allowed to adhere for 24 h; incubated with red-fluorescent MagSiNs equivalent to the dose of Dox-MagSiNs that would deliver 20 nM Dox·HCl; and exposed 24 h to a magnetic field (27-35 Gauss). The medium was then replaced, and the cells were incubated with the nanoparticles for different times. To track the endocytic pathway, the cells were labeled 30 minutes with Lysoview DND Green 50 nM (Life Technologies Ltd). The wells were rinsed twice with 400 μL of 1×PBS. Lastly, cells were fixed in 4% buffered parafomaldehyde, stained with DAPI, and the slides were taken to be imaged with a NikonU fluorescent microscope with a 10× lens and the green, blue, and red fluorescent channels.

### Animal Experiments

### Biodistribution

Balbc/J mice were purchased from Jackson Labs (ME). Five time cohorts (1 h, 4 h, 8 h, 24 h, and 48 h post-MagSiNs injection) (n=3-4/cohort) were created. The mice were restrained and 200 μL of 10 mg/mL MagSiNs in sterile 1×PBS was injected through the tail vein. The mice were ˜7 weeks old. The control mice had only sterile 1×PBS injected into them. At each time point, the mice were sacked by anesthetizing them with isofluorane followed by cervical dislocation. The blood was drawn by cardiac puncture. The brain, heart, lungs, liver, kidney, spleen, and fecal pellets in the large intestine were harvested and fixed in 4% buffered para-formaldehyde. The samples were all stored at 4° C. All procedures were approved by the Institutional Animal Care and Use Committee at the University of Notre Dame and were conducted in accordance with the guidelines of the U.S. Public Health Service Policy for Humane Care and Use of Laboratory Animals.

### Tumor Induction and Tracking in Mice

Tumor formation was induced by injection of red fluorescent triple negative MDA-MB-231 breast cancer cells (MDA-231-RFP) into the right rear flank of female Foxn1^{nu/nu} athymic mice (Jackson Labs). Specifically, 500,000 to 1,000,000 MDA-231-RFP cells were suspended in sterile PBS and injected in each mouse. MDA-MB-231 cells induce a human triple negative primary tumor that grows into the mammary tissue with the possibility of metastasis to multiple organs. The MDA-MB-231 tumors were allowed to grow to at least 5 mm in diameter over 6 weeks. The primary tumor growth in the mouse tissue and any metastases were monitored longitudinally using RFP fluorescence. Any metastases were quantified at experiment termination by counting visible tumors on the surface of the organs after H&E staining to differentiate tumor from healthy tissue. The tumors had a median doubling time of ˜5-6 days, which allowed for a 3-4-week therapeutic window to evaluate anti-tumor responses.

Dox MagSiNs (50 mg/kg) were injected as a single bolus through the tail-vein. After Dox-MagSiNs injection, the mice were exposed to a magnetic field gradient for 24 hr through an array of neodymium magnets below the mouse cage that generated a magnetic field of about 25-30 Gauss around the whole mouse body. After 24 hr of magnetic field exposure, the mice were imaged using in vivo fluorescence. Red fluorescence was used to track the primary tumor and metastatic sites. The Dox MagSiNs had Alexa Fluor 750 near-IR fluorescent dye embedded in the silica shell of the MagSiNs. Fluorescence from the Alexa Fluor 750 dye was used to track the Dox MagSiNs in vivo and to detect and measure co-localization of the Dox MagSiNs with the tumor cancer cells.

### Inductively Coupled Plasma-Optical Emission Spectroscopy (ICP-OES)

Selected organs (liver, spleen, kidney, lung, heart, brain, intestine, skin, and blood), were dissected from three mice in each group, dried overnight in an oven at 37° C., massed, and digested in aqua regia (3 HCl: 1 HNO₃) for 24 h. The mass of Fe, Co, Si in each sample was measured using ICP-OES (Optima 8000, Perkin Elmer, Waltham, MA, USA). Calibration curves were created by diluting certified standard Fe, Co, and Si solutions (VWR, Radnor, PA, USA).

### Histology

The fixed organs were sliced, then the slice was rinsed with PBS, dehydrated in a graded series of ethanol solutions, embedded in paraffin, sectioned to 4 μm, and stained with hematoxylin and eosin. Stained tissue sections were imaged by transmitted light microscopy (Eclipse ME600, Nikon Instruments, Melville, NY, USA) at 1000× magnification and interpreted by a medical pathologist.

### Example 2

### Physical Characterization of MagSiNs

The MagSiNs nanoparticles were made of a novel core-shell composition of cobalt ferrite and silica (CoFe₂O₄—SiO₂), in which the relatively high moment CoFe₂O was used to enhance the magneto-electric coefficient. Previous studies have focused on proof-of-concept experiments with no consideration for scale-up manufacture or batch to batch consistency. In this study, despite the novelty of the core-shell NPs for on-demand drug release, the wet synthesis yielded

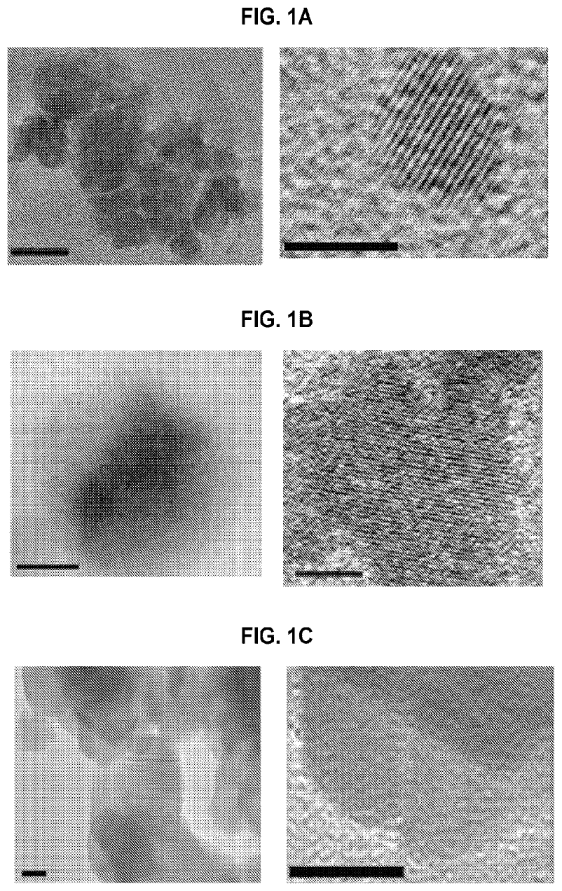

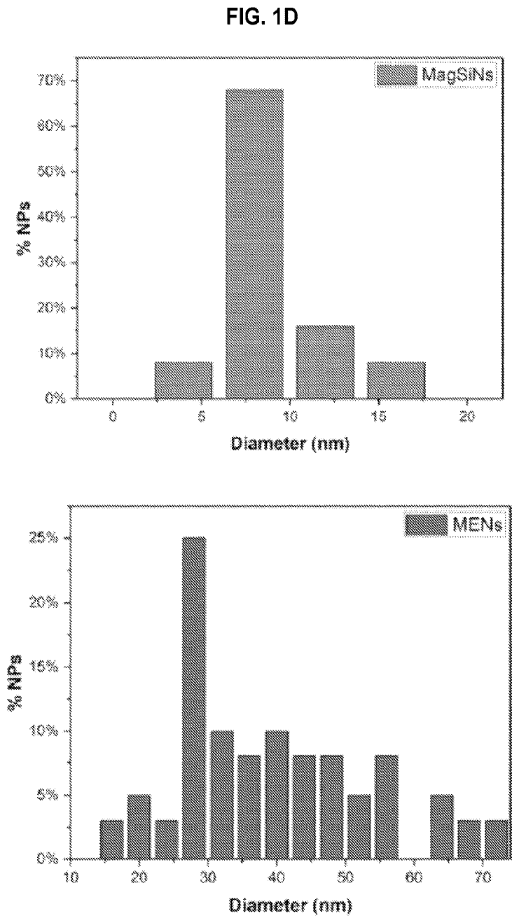

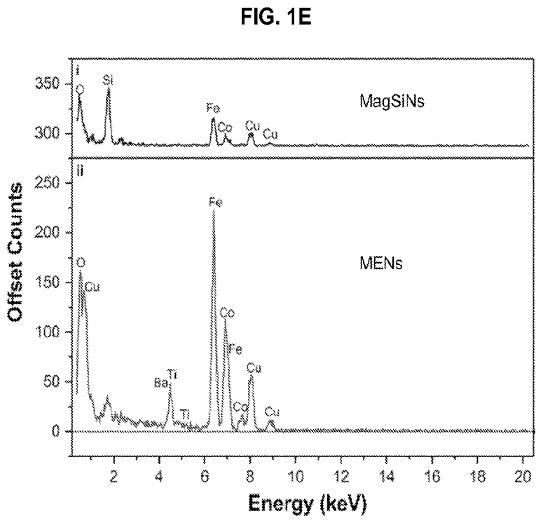

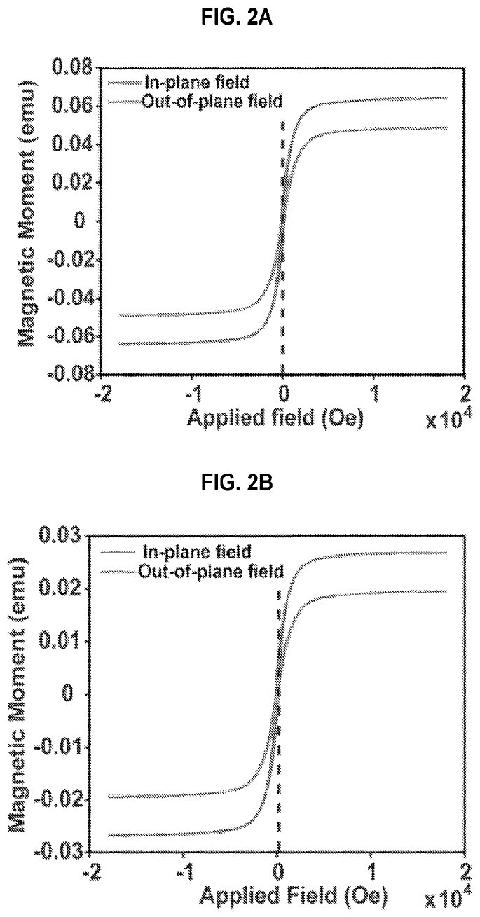

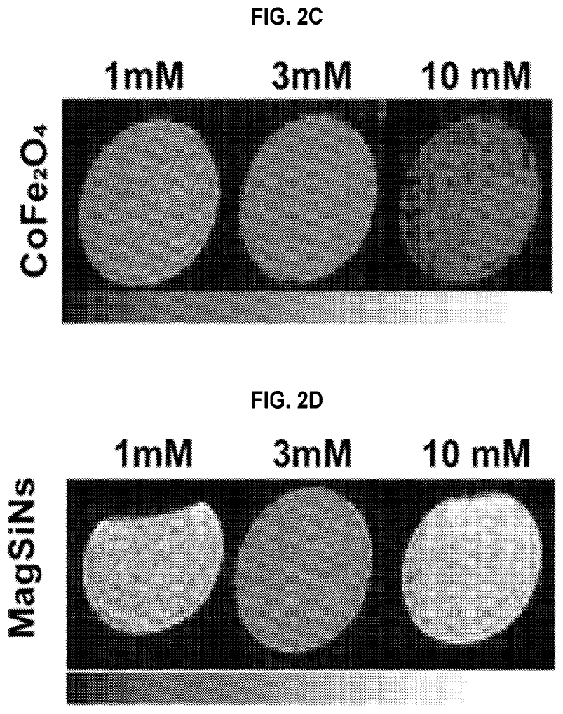

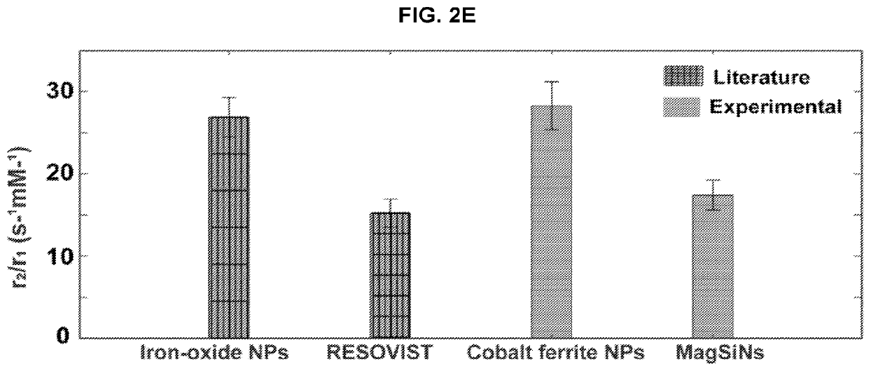

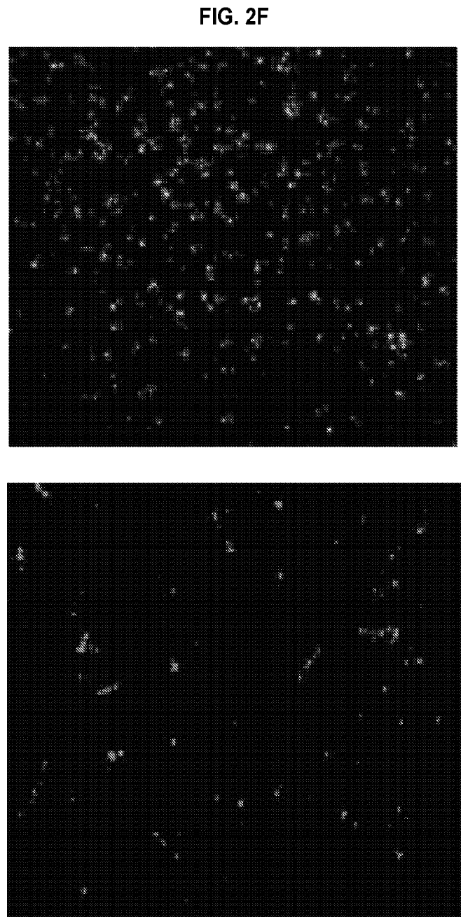

NPs in the 0.1 kg range as opposed to the current state of the art that yield only milligrams of nanomaterials. A typical transmission electron microscopy image of the fabricated CoFe₂O₄ is shown in FIG. 1A. Transmission electron microscopy images showing CoFe₂O₄ nanoparticle cores capped in a fused silica shell silica is shown in FIG. 1B. The crystal lattice of CoFe₂O₄ nanoparticles was clearly visible at 150,000-200,000× magnifications, as was the silica shell. The silica-shell thickness was 1.51±0.94 nm. The lattice spacing on the CoFe₂O₄ nanoparticle cores was 0.865±0.038 nm, which is consistent with cubic spinel crystal structure from the literature (FIG. 1A). The MENs had a barium titanate shell with a shell thickness varying from 10 nm to 40 nm (FIG. 1C). From the TEM image analysis, the MagSiNs diameter was 6.71±2.48 nm (FIG. 1D, top). From the TEM image analysis, the MENs diameter was 37.1±13.9 nm (FIG. 1D, bottom). The elemental composition of the MagSiNs was confirmed through energy-dispersive spectroscopy to be Co, Fe, O, and Si as shown in FIG. 1E. The elemental composition of the MENs was confirmed through energy-dispersive spectroscopy to be Co, Fe, O, Ba, and Ti as shown in FIG. 1E. The EDXS peak heights are consistent with the stoichiometry of Co:Fe=1:2. The magnetic hysteresis loops of the sample cobalt ferrite NPs and MagSiNs were measured using a Microsense EV7 vibrating sample magnetometer (VSM) (FIG. 2A-B). During the operation of the VSM, the CoFe₂O₄ or the MagSiNs sample were vibrated between pick up coils. A DC external field from an electromagnet magnetized the sample. The resulting oscillating magnetic field from the sample induced an alternating emf which was proportional to the total magnetic moment of the sample. Sweeping the external magnetic field and measuring the resulting magnetic moment gave the magnetic hysteresis loop of the sample. The nanoparticles were deposited on 1×1 cm² silicon dies and measured in the VSM. The magnetic field was swept from −18 to 18 kGa. The contribution of the silicon and silicon dioxide was removed by removing the slope of the curve using the slope from 10 kGauss to 18 kGauss where the samples are saturated. The magnetic properties of the samples are summarized in Table 1 below. The saturation magnetization (emu/g) of MagSiNs was 53.20 emu/g (in-plane) and 3.70 emu/g (out-of-plane) in comparison to Cobalt Ferrite which was 72.14 emu/g (in-plane) and 39.07 emu/g (out-of-plane). The saturation magnetization is the point at which the material obtains maximum alignment with the applied magnetic field. This corresponds to the maximum magnetic moment that can be obtained. The magnetostrictive coefficient is only applicable until the saturation magnetization because the material reaches its maximum strain and cannot continue to elongate at this point. The maximum elongation also corresponds to a 90°-Bloch wall and domain rotation over the total volume of material. In other words, the saturation magnetization varies between materials and occurs when their domains have all been aligned. Any increase of the applied magnetic field past the saturation point would then not affect the material because its domains could not be further aligned. When the applied magnetic field is removed and no stress is applied to the material, the magnetostriction exhibits some hysteresis. This occurs because the material is still magnetized when the magnetic field is removed, which results in nonzero strain in the material. Magnetostrictive materials are used to convert electromagnetic energy into mechanical energy and vice versa. The magnetic field or force applied would create a strain in the material. The VSM hysteresis loop confirmed the suitability of the magnetostrictive properties of the cobalt ferrite core in the MagSiNs to produce vibrations in the presence of an alternating magnetic field, which can then induce charge polarization on the piezoelectric silica shell of MagSiNs. VSM measurements were indicative of favorable magnetostrictive properties that is crucial for the magnetoelectric effect and ON-Demand drug release from MagSiNs.

TABLE 1
Comparison of Magnetic Properties of Cobalt-
Ferrite Cores and MagSiNs from VSM measurements

| | | | Total | | |
|---|---|---|---|---|---|
| | | | Magnetic | | |
| | | | Moment at | | Saturation |
| | In-Plane or | Coercivity | Saturation | Mass | Magnetization |
| Sample | Out-of-Plane | (Gauss) | (emu) | (g) | (emu/g) |
| Cobalt Ferrite | In-plane | 120.68 | 1.01 × 10⁻¹ | 1.40 × 10⁻³ | 72.14 |
| Cores | Out-of-plane | 112.396 | 5.47 × 10⁻² | 1.40 × 10⁻³ | 39.07 |
| MagSiNs | In-plane | 125.564 | 2.66 × 10⁻² | 5.00 × 10⁻⁴ | 53.20 |
| | Out-of-plane | 119.742 | 1.85 × 10⁻² | 5.00 × 10⁻³ | 3.70 |

The MagSiNs size distribution fell within 4.3 nm to 9.1 nm with peak diameter centered around 6.7 nm. This size range is ideal for materials being designed with in vivo applications in mind. Nanomaterials that are >5 nm avoid being filtered out through the renal system. The narrow size range between 4.3 nm to 9.1 nm size also makes it easier to model nanoparticle distribution in a flowing fluid. The >5 nm MagSiNs can also circulate multiple times through the blood circulatory system allowing higher probability of localizing to target tissue.

### Example 3

### Characterization of Drug Nanocarrier Properties

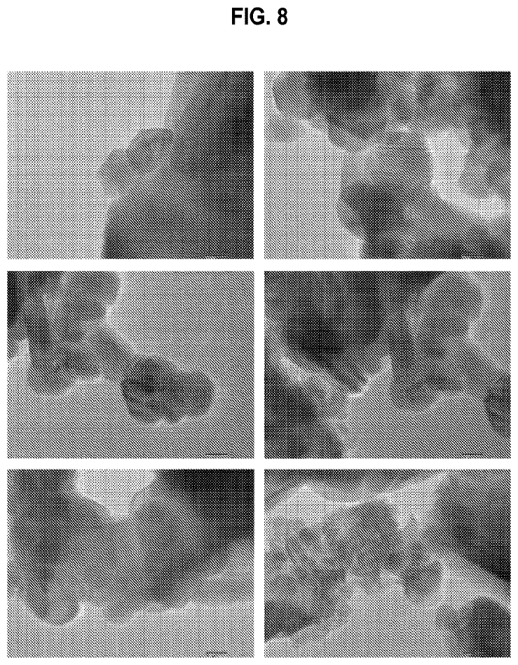

In Table 2 below, the stability of the drug payload on the nanocarriers and the release kinetics of different stimuli-responsive drug delivery mechanisms is shown. Several drug-loading mechanisms were explored. Reproduction of electrostatic loading and release of fluorescein isothiocyanate (FITC-a Dox proxy) from cobalt ferrite-barrium titanate core-shell magneto-electric nanoparticles (MENs) was attempted (FIG. 8). A previous technique was also modified to encapsulate cobalt ferrite cores in a piezoelectric fused-silica shell (MagSiNs). Ester linkers were used to immobilize FITC on MagSiNs to explore mimicking acid hydrolysis of ester bonds in lysosomes to release the active form of the drug from MagSiNs. A 2-carbon long ethyl linker and a 4-carbon long butyl linker between the ester bond and FITC were used to investigate how linker length affects FITC release. The immobilization of FITC on MagSiNs through the formation of an amide bond was also explored with carboxylate groups on the surface of the MagSiNs followed by low frequency (50-100 Hz) AC magnetic field to induce release of FITC from MagSiNs.

The electrostatic loading of FITC on MENs was not stable and it leached the fluorescent payload constantly. 100% of the fluorescent payload was released in 1 h of a 100 Hz alternating magnetic field at 27-30 Gauss. However, 84% of the fluorescent payload was released in 1 h even without the magnetic field. Additionally, the barium titanate shell was not of reproducible thickness leading to a broad distribution in the size and shape of CoFe₂O₃—BaTiO₃ core-shell NPs. These two factors may lead to inconsistent drug loading, unpredictable drug release, and unwarranted off-target toxicity when transitioned to an in vivo setting.

For a second technique, the acidic environment of cancer cells was exploited to trigger the release of drugs from MagSiNs. By increasing the length of the carbon spacer between the ester bond and the drug molecules, it is possible to control the ester hydrolysis rate and thereby the drug release rate from the nanocarrier. The ester hydrolysis rate is higher at acidic pH and therefore drug release is expected to accelerate in the acidic environs of lysosome-like organelles as well as acidic extra-cellular matrix of cancer cells. The release of FITC at two pH values—pH 4.75 (MES buffer) and pH 7.2 (PBS buffer)—was tested. The ester bond with the butyl linker released the payload at 0.98%/hour at pH 7.2 and at 2.1%/hour at pH 4.75. This translated to a cumulative payload release of ˜50.3%±3% at pH 4.75 over a 24 h time period as opposed to 23.6%±1.2% at pH 7.2 for 24 h. However, for the ester bond with the ethyl linker to the payload, there was no significant pH dependence. The ester bond with the ethyl linker released the payload at 2.5%/hour at pH 7.2 and at 2.8%/hour at pH 4.75. This translated to a cumulative payload release of ˜66.2%±3.4% at pH 4.75 over a 24 h time period as opposed to 59.9%±2.5% at pH 7.2 for 24 h.

TABLE 2
Kinetics of payload release as a function of drug-release mechanisms.
The payload RBITC was used as proxy for drug molecules

| | | | | | | |
|---|---|---|---|---|---|---|
| | | 100 Hz | | | | |
| | | Magnetic | | | | Rate of |
| | | Field | | Observation | % Payload | Payload |
| Nanoparticle | Linker | Strength | | Time Point | (FITC) | (FITC) |
| Type | Chemistry | (Gauss) | pH | (h) | Released | Released |

Drug Loading through Covalent Bond

| | | | | | | |
|---|---|---|---|---|---|---|
| MagSiNs | Isothiocyanate | 27-35 | 7.2 | 0.5 h | 79.9% ± | 159.8% per |
| (Cobalt ferrite | to amine | | | magnetic | 0.2% | hour |
| core and | | | | stimulation | | |
| piezoelectric | | 27-35 | 7.2 | 1 h magnetic | 78.9% ± | 78.9% per |
| silica shell) | | | | stimulation | 0.1% | hour |
| | | 27-35 | 7.2 | 1.5 h | 79.9% ± | 53.3% per |
| | | | | magnetic | 0.2% | hour |
| | | | | stimulation | | |
| | | 27-35 | 7.2 | 3 h magnetic | 90.1% ± | 30% per |
| | | | | stimulation | 0.3% | hour |
| | | 27-35 | 7.2 | 8 h magnetic | 100% ± | 12.5% per |
| | | | | stimulation | 0.3% | hour |
| | | 0 (control) | 7.2 | 48 h | 3.6% ± | 0.075% per |
| | | | | | 1.2% | hour |

Drug Loading through Covalent Ester Bond that is acid labile

| | | | | | | |
|---|---|---|---|---|---|---|
| MagSiNs | Ethyl-acetate | No | 7.2 | 24 h | 59.9% ± | 2.5% per |
| (Cobalt ferrite | ester bond | magnetic | | | 2.5% | hour |
| core and | | field | | | | |
| piezoelectric | | No | 4.75 | 24 h | 66.2% ± | 2.8% per |
| silica shell) | | magnetic | | | 3.4% | hour |
| | | field | | | | |
| MagSiNs | Butyl-acetate | No | 7.2 | 24 h | 23.6% ± | 0.98% per |
| (Cobalt ferrite | ester bond | magnetic | | | 1.2% | hour |
| core and | | field | | | | |
| piezoelectric | | No | 4.75 | 24 h | 50.3% ± | 2.1% per |
| silica shell) | | magnetic | | | 3% | hour |
| | | field | | | | |

Drug Loading through Hydrophobic Encapsulation

| | | | | | | |
|---|---|---|---|---|---|---|
| MENs (Cobalt | Electrostatic | 27-35 | 7.2 | 1 h magnetic | 100% ± | 100% per |
| ferrite core and | loading in | | | stimulation | 1.5% | hour |
| piezoelectric | glycerol mono- | 0 | 7.2 | 1 h | 84% ± | 84% per |
| barium titanate | oleate layer on | | | | 2.1% | hour |
| shell) | MENS | | | | | |

In either case, the first-order kinetics of drug release and the ensuing compartmentalization rate of the drugs to the cancer cell would have been too slow to overcome the chemoresistitve mechanisms of the cancer cells. Additionally, similar to electrostatic loading, there was a steady leaching of the fluorescent payload into the solution from the nanocarrier even at neutral pH, which again made it unsuitable to avoid off-target toxicity.

In contrast, when the FITC payload was linked to the MagSiNs by means of an amide linker, the total free payload observed in solution after 24 h was 3.6% which remained a constant over 4 days. In the presence of the 27-30 Gauss, 100 Hz, AC magnetic field, up to 80% of the payload was released 30 minutes post-exposure to the AC magnetic field, which is the much preferred near-instantaneous release of payload from the nanocarriers. 90% cumulative release of payload and ˜100% cumulative release of payload was measured at 3 h and 8 h post-exposure to the AC magnetic field.

The near instantaneous release profile of the payload from the nanocarrier means that cancer cells will be exposed to the full dose of the drugs in a short burst, which is favorable for compartmentalization of drugs to the cancer cells such that the drugs can exert their anti-cancer effect before being neutralized by the chemoresistant mechanisms of cancer cells. The lack of leaching of the payload from the MagSiNs surface in the absence of an AC magnetic field bodes well for negating off-target toxicity of such cancer therapeutics such as Doxorubicin. Therefore, Dox·HCl covalently immobilized on MagSiNs followed by AC magnetic field release of Dox·HCl was further investigated as a label-free, ON-Demand chemotherapeutic delivering nanocarrier.

### Example 4

### Characterization of the Magnetoelectric Properties of MagSiNs

VSM measurements (FIG. 2A-B) showed magnetization and demagnetization repeatedly for both cobalt ferrite nanoparticles and MagSiNs, highlighting the magnetostrictive property of the cobalt ferrite cores which is necessary to induce the magneto-electric effect in the piezoelectric shell. This was tested by measuring the zeta-potential of the MagSiNs in the presence of different magnetic-field strengths. The zeta potential range of cancer cells listed in Table 2 from a literature survey was ˜−14 mV to −25 mV (A2780), 12.8±2.8 mV (PC-3), and −15 to −18 mV (MDA-MB-231). For HUVECs, which are derived from human umbilical vein, the zeta potential has previously been measured at 12.8±0.56 mV. Although the zeta potential of the control cells (HUVECs) and the cancer cells (A2780, PC-3, MDA-MB-231) fell within the same dynamic range, the stiffness of HUVEC cell membranes are 10-fold to 1000-fold higher than the cancer cell membranes which were highly pliant (Table 2). Interestingly, the zeta potential of MagSiNs can be tuned from −6.8 mV to −25.6 mV by using a magnetic-field from 0 Gauss to 256 Gauss (Table 3). This is due to the magnetostriction of the core in the magnetic field, which in turn deforms the piezoelectric shell to varying degrees, resulting in an increase in charge presentation on the MagSiNs surface. The MagSiNS did exhibit low zeta potential values. These values led to flocculation (reversible) over time at high concentrations (1% w/v) of MagSiNs, but not aggregation (irreversible). The MagSiNs could always be resuspended by vortexing.

However, the kinetics of flocculation are also influenced by the initial concentration of nanoparticles in solution. Because a 1000-fold to 10,000-fold less than 1% w/v was typically used, there were no issues with the stability of dispersion. For the magnetic field of 27-30 Gauss, the zeta-potential of the MagSiNs was between −11 mV to −15 mV. This pointed to the ability of the MagSiNs to match the membrane potential of cancer cell which in turn will enable them to interact with the cell membrane for prolonged durations without repulsion, and to actively electronanoporate across the cancer cell membranes. This, in theory, makes MagSiNs ideal drug nanocarriers.

TABLE 3
Magnetic field dependent zeta potential on the surface of MagSiNs

| MagSiNs | | | | |
|---|---|---|---|---|
| Magnetic | | Cells | | |
| Field | [symbol] Zeta | | * Zeta | ^{¥} Cell |
| Strength | Potential | Cell | Potential | Stiffness |
| (Gauss) | (mV) | Lines | (mV) | (kPa) |
| 0 | −6.8 ± 0.66 | HUVEC | −12.8 ± 0.56 | 10-11 |
| 30 | −11.2 ± 1.5 | A2780 | −14 to −25 | 1.25 ± 0.5 |
| 40 | −13.4 ± 2.1 | PC-3 | −12.8-2.8 | 0.4 ± 0.05 |
| 265 | −25.6 ± 3.0 | MDA-MB-231 | −15 to −18 | 0.018 to 0.04 |

[symbol]

Experimentally determined in laboratory.
* Cell zeta potentials determined from the peer-reviewed literature survey.
^{¥} Cell stiffness determined from the peer-reviewed literature survey.

### Example 5

### Characterization of Image Contrast Properties of MagSiNs

The magnetic resonance image (MRI) contrast enhancing efficacy of the synthesized spherical cobalt-ferrite and fluorescent MagSiNs nanostructures (T₂ agent) is characterized by its relaxivity coefficient (r₂), which is related to T₂ through Equation (1):

$$1 / {T}_{2} = 1 / {T}_{2} + {r}_{2} C , \qquad ( 1 )$$

where C is the contrast agent concentration, T₂ is the observed relaxation time in the presence of cobalt ferrite nanostructures, and T₂⁰ is the relaxation rate of pure water. In the equation, T₂ becomes shorter when the concentration (C) increases, while r₂ is the relaxivity coefficient. From the given equation, it reveals that as the concentration increases, the MRI image appears darker and contrast agents having a higher r₂ value require small concentration increments. In other words, unlike T₂, which depends on concentration, r₂ is a concentration-independent term. A contrast agent with a large r₂ value can shorten T₂ drastically with a smaller concentration increment. T₁-T₂ averaged MRI scans were taken with reference to the concentration of CoFe₂O₄ nanoparticles. As expected, CoFe₂O₄ nanoparticles showed a T₂-weighted effect with concentration-dependent enhancement of negative contrast in the image (FIG. 2C). However, MagSiNs with the silica shell showed a T₂-weighted, negative contrast effect of only until 3 mM CoFe₂O₄ concentration. However, this still demonstrated the efficacy of MagSiNs as MRI contrast agents (FIG. 2D).

The efficacy of NPs as a contrast agent for MRI is related to their relaxivity values (r₁, r₂). The ratios of relaxivities are reported with respect to the total molarity of iron and cobalt (i.e., s⁻¹ mM⁻¹ Fe) (FIG. 2E). The ratio of transverse/longitudinal relaxivity (r₂/r₁) for iron-oxide NPs and commercially available T₂-contrast agent RESOVIST™ were compared to the CoFe₂O₄. The cobalt ferrite NPs had r/r₂ values of 26.9±2.4 s⁻¹ mM⁻¹ which was comparable to the r₂/r₁ values of iron oxide NPs 28.3±2.9 s⁻¹ mM⁻¹. However, the MagSiNs had an r₂/r₁ value that was significantly lower at 15.2±1.7 s⁻¹ mM⁻¹ but still comparable to the RESOVIST™ contrast agent at 17.4±1.8 s⁻¹ mM⁻¹. This was interesting because CoFe₂O₄ nanoparticles showed a T₂-weighted effect with higher negative contrast at increasing concentrations of CoFe₂O₄ up to 10 mM, which was the highest concentration tested. MagSiNs showed T₂-weighted image contrast up to 3 mM concentration iron but exhibited Ti-weighted image signal enhancement at 10 mM MagSiNs. This concentration-dependent T₁ or T₂ enhancement by MagSiNs was very similar to previously published research with RESOVIST™, which initially showed Ti-weighted signal enhancement immediately after administration to the patient but showed T₂-weighted image contrast at approximately 10-15 min post administration and clearance.

After demonstrating the ability to reproducibly encapsulate the cobalt ferrite core in a silica shell and characterizing the magnetic and MRI properties of the core vs. the core-shell nanoparticles, previously optimized protocols were used to incorporate fluorophores within the silica shell (volume-loading) to gain fluorescent modality without altering the surface properties of the MagSiNs. Without covalent attachment, dye molecules weakly associated with the porous structure of the amorphous silica leak into the surrounding environment. This is the most common problem associated with the integration of organic dyes into silica nanoparticles. Many studies have attempted to resolve this problem by using coupling agents and chemical binding. However, the low intensity in fluorescence and resulting low sensitivity of the organic dyes used limited their applications. By volume-loading the fluorophores into the silica shell, the fluorescence signal from fluorophores will also be impervious to solvent effects and pH effects. RITC or FITC, which when linked to aminopropyltriethoxysilane, were readily co-precipitated with tetraethoxysilane into the PVP mesh surrounding the cobalt ferrite nanoparticles to yield a discrete fluorescent silica shell by the modified Stöber method. This resulted in either red-fluorescent or green-fluorescent MagSiNs with steady fluorescent signals which enabled tracking of the MagSiNs during in vitro studies (FIG. 2F). Therefore, synthesis of MagSiNs with dual-modalities of detection (MRI and fluorescence) was demonstrated. Fluorescence modality is suitable for tracking MagSiNs in vitro, while MRI modality is suitable for tracking MagSiNs in vivo.

### Example 6

### Characterization of Cytocompatibility, Biocompatibility, and Biodistribution of MagSiNs

For suitability as a drug carrier, it was important to assess the cytocompatibility and biocompatibility of MagSiNs. Cytocompatibility was assessed against HUVEC cells, which is model cell line for blood vessels and is utilized extensively to assess the cytocompatibility of intravenously-delivered therapeutics. Biocompatibility was assessed in immunocompetent Balbc/J mice.

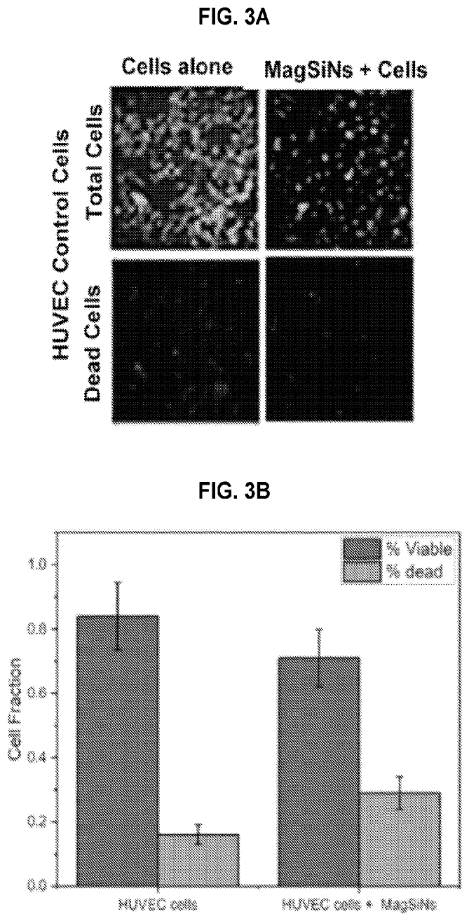

HUVEC cells were incubated with 0.116 μg MagSiNs for 48 h. A Live/Dead assay using calcein AM ester/propidium iodide was used to differentiate live cells from dead cells (FIG. 3A). Statistically, there was no difference in cell viability of control HUVECs grown in complete growth medium vs. HUVECs grown in media supplemented with MagSiNs. The control groups had 84±10.5% live cells and viable cells in MagSiNs-exposed test cell group had 71±8.9% live cells (FIG. 3B).

For the biocompatibility assessment, Balbc/J mice (n=4) were each injected with 10 mg/kg MagSiNs through the tail-vein. There was a mice cohort for each time point (1 h, 4 h, 8 h, 24 h, 48 h) and each cohort was sacked at that time point post-injection of MagSiNs. At the endpoint, the brain, heart, lungs, liver, kidney, spleen, and fecal pellets were collected and fixed in 4% buffered paraformaldehyde for further analysis. At least 100 μL to 500 μL of blood was harvested per mouse by cardiac puncture and stored between 2-8° C. The collected tissues were scored for inflammation by H&E staining of histology sections (FIG. 3C). Additionally, a known mass of each tissue was digested and analyzed by ICP-OES for quantifying the biodistribution of MagSiNs longitudinally.

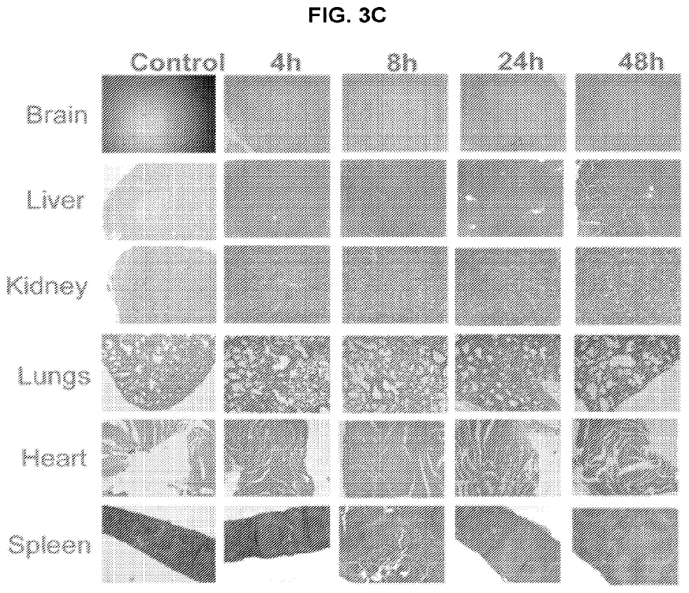

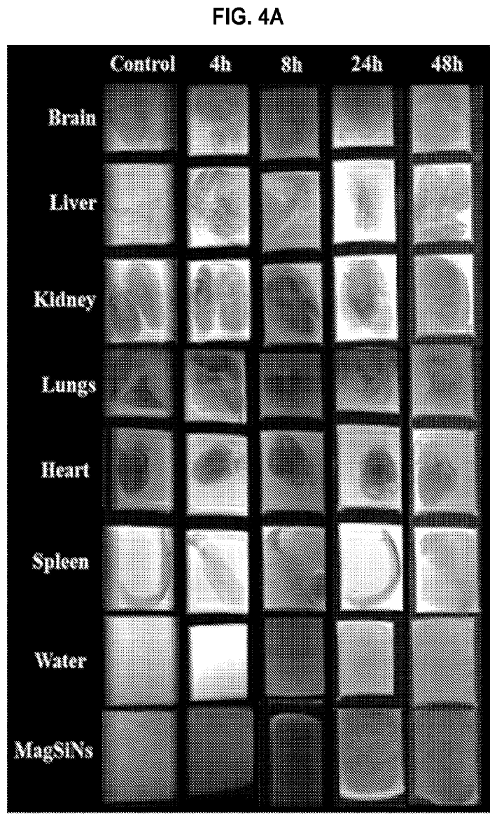

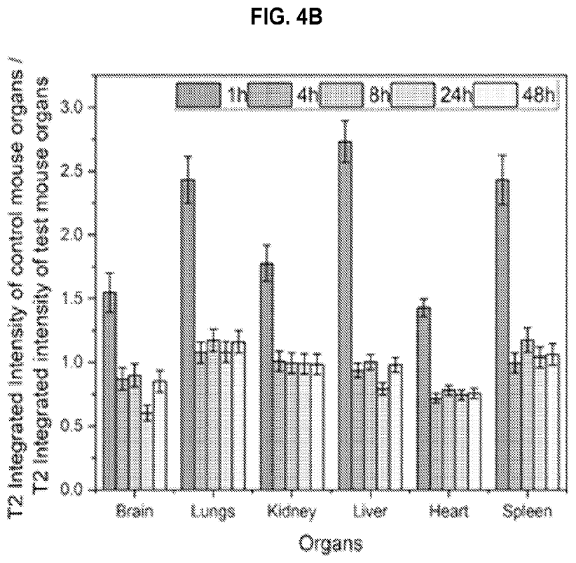

Histology scoring of tissue samples up to 48 h post-exposure to MagSiNs did not indicate any inflammation in comparison to the control mice cohort. Ex vivo MRI imaging was acquired using T₁ and T₂ scans on the Bruker desktop 1T MRI (FIG. 4A). To determine if the biodistribution trended towards bioclearance or bioaccumulation, the ratio of the integrated image intensity of the T₂-weighted scan of the control mice tissue against that of the integrated image intensity of the T₂-weighted scan of the mice tissue from each timepoint cohort was calculated (FIG. 4B). This ratio had an exponential negative slope indicating insignificant non-specific accumulation of MagSiNs in mouse tissue and organs. These results indicate good bio-clearance of MagSiNs from organs 48 hr post-injection.

Interestingly, the ICP-OES analysis of the fecal pellet showed a sinusoidal curve with peak MagSiNs at 8 h and 48 h, which indicated clearance of the MagSiNs through the GI tract (Table 4). ICP-OES of the fecal pellets determined the control mice (i.e., no MagSiNs exposure) had a baseline signal of 10.7±2.0 Fe (ppb) per milligram of sample. There was a statistically significant increased amount of Fe in the fecal pellet of mice injected with MagSiNs at the 4 h (36.2% higher) and 24 h (50.4% higher) post-injection marks. This indicated that the MagSiNs were indeed being cleared out through the GI tract and not accumulating in vivo.

TABLE 4
Inductively coupled plasma-optical emission spectra (ICP-OES)
from iron (Fe) per milligram of intestinal fecal pellets

| Time post- | | Standard | Difference |
|---|---|---|---|
| injection | Average Fe | Deviation | from |
| (hr) | (ppb)/mg | Fe (ppb)/mg | control |
| 4 | 14.3 | 2.5 | 1.3 |
| 8 | 9.0 | 2.3 | 0.8 |
| 24 | 15.8 | 2.5 | 1.5 |
| 48 | 10.5 | 2.4 | 1.0 |

The combined in vitro and in vivo testing demonstrate the cytocompatibility and biocompatibility of the disclosed MagSiNs. The biodistribution studies utilized T₂-weighted MRI scans and ICP-OES to demonstrate effective clearance of the MagSiNs through the GI tract without any non-specific accumulation in tissues over a 48 h period post-injection. The biodistribution results, combined with the in vitro and in vivo biocompatibility results confirmed the suitability of utilizing MagSiNs as drug nanocarriers.

### Example 7

Anti-Cancer Efficacy of Doxorubicin Released from Dox-MagSiNs

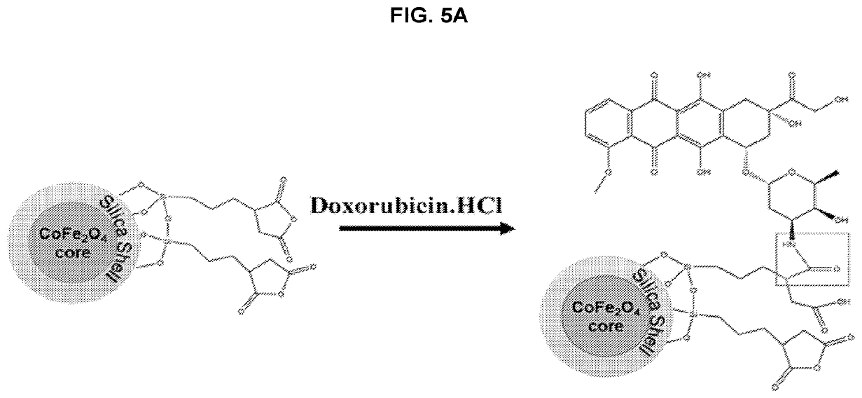

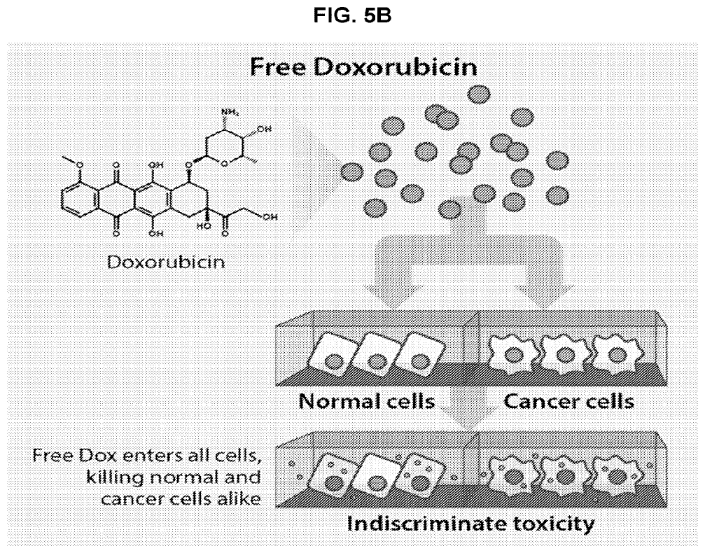

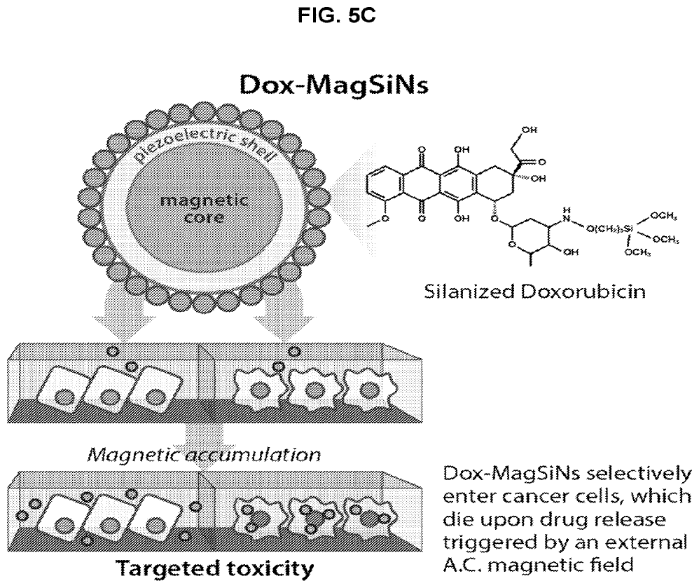

A schematic illustration of the addition of free Dox and silanized-Dox conjugated to MagSiNs to normal and cancer cells is shown in FIG. 5A-C. The Dox-MagSiNs were incubated with the cells while exposed to a permanent magnet, followed by drug-release in an alternating electromagnetic field.

Dox-MagSiNs were added to the metastatic cancer cells from ATCC (MDA-MB-231, PC-3, A2780) or normal HUVEC cells, exposed to a unidirectional magnetic field (24-50 Gauss) for 24 h, and then Dox·HCl release was triggered in a 100 Hz alternating electromagnetic field of the same strength to demonstrate that the ON-Demand release of Dox·HCl activates its cytotoxic activity. Simultaneously, as a control group, the same cell lines were also treated with free drugs alone to compare the anti-cancer efficacy of free drug formulations (Dox·HCl) to drug formulations delivered on MagSiNs (Dox-MagSiNs).

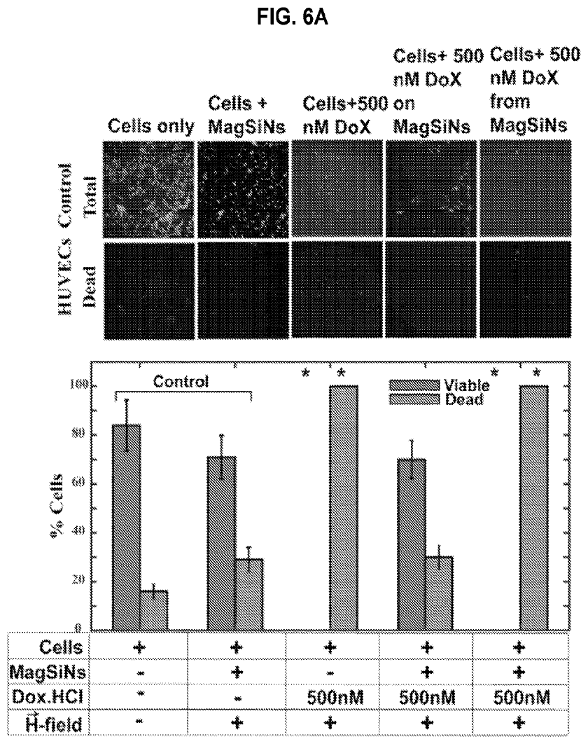

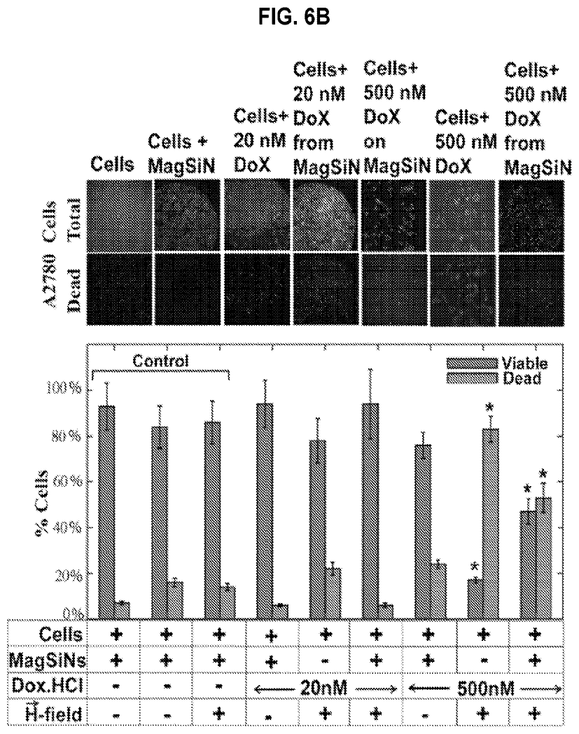

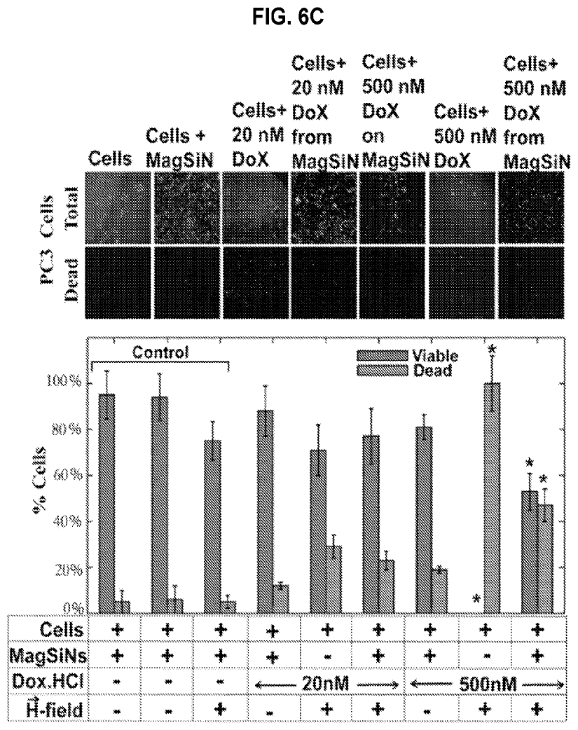

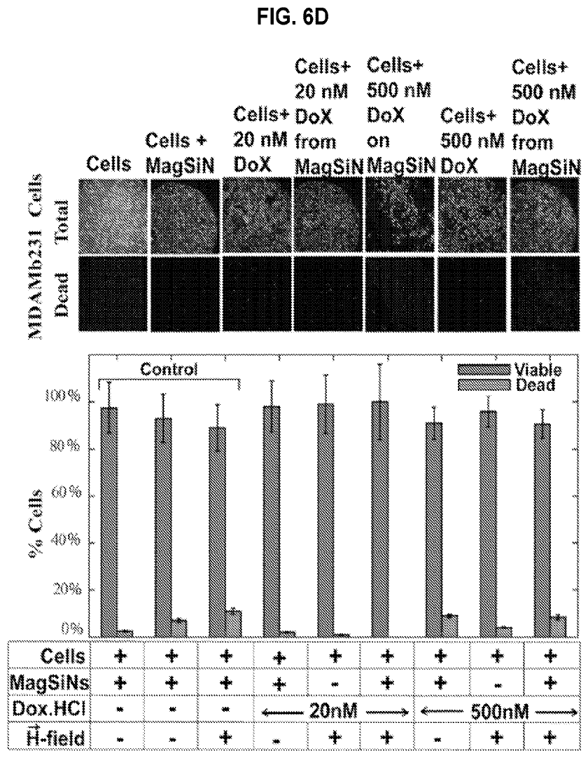

Viability assays (FIG. 6A-D) were executed for 3 total sample groups: a) free Dox, b) Dox-MagSiNs, and c) cell-culture media. The concentration of Dox conjugated to the Dox-MagSiNs was matched by the concentration of free drug formulations (20 nM Dox·HCl or 500 nM Dox·HCl). Viable cell populations of treated and untreated control cancer cell lines and treated and untreated normal cell lines were determined at the end of each run using a calcein-AM/propidium iodide assay to determine total cells and viable cell populations. The calcein-AM assay is based on the conversion of the cell permeant non-fluorescent calcein-AM dye to the fluorescent calcein dye by intracellular esterase activity in live cells. Propidium iodide (PI) is membrane impermeant and therefore does not enter viable cells with intact membranes. When PI does gain access to nucleic acids and intercalates, its fluorescence increases dramatically and is therefore used to identify dead cells. Calcein-Am and PI can be used separately or together to assess cellular viability or cell death, respectively. Statistical analysis using a paired t-test (α=0.05) was used to confirm any significant difference in cell viability when comparing controls with cells exposed to Dox from Dox-MagSiNs.

In this study, for each sample set (control group or test group), the total number of cells with green fluorescence and the total number of cells with red fluorescence were counted as separate datasets. It was determined that the green cells were representative of total cells in the image while the red cells indicated only the dead cells in the image. For each sample set, the percent dead-cells and percent live-cells was calculated. The percent live-cells was used as a measure of viability. A paired t-test under the assumption of comparing two-samples with equal variances and a >=0.05 was used to determine the statistical significance of the test-groups in comparison to the control groups.

### HUVEC Control Cells (FIG. 6A)

The control group for HUVECs consisted of three sample-sets. HUVECs were grown in cell culture medium or were cultured in growth medium infused with MagSiNs equivalent to 500 nM Dox from Dox-MagSiNs. The control sample-sets cultured with MagSiNs were further split into two groups with one group exposed to no permanent magnetic field while the second group was exposed to 24 h of permanent magnetic field. Addition of free Dox·HCl at 20 nM and 500 nM resulted in 100% HUVEC death. However, when Dox-MagSiNs with 500 nM equivalent of Dox·HCl was incubated with HUVECs, the viability of the exposed HUVECs was not statistically different from the control sample-sets. Furthermore, when either 20 nM or 500 nM equivalent dose of Dox release was triggered using an AC magnetic field from the Dox-MagSiNs, this again resulted in 100% HUVEC cells death, similar to the free Dox·HCl doses.

The fact that for normal, control, HUVECs, 100% cell death was observed after exposure to 20 nM or 500 nM Dox·HCl in its free form or after release from MagSiNs indicates Dox activity is retained after release from Dox-MagSiNs. Another important result was the complete biocompatibility of Dox-MagSiNs to HUVECs in the absence of an AC magnetic field to trigger the release of the Dox·HCl from Dox-MagSiNs.

### A2780 Ovarian Cancer Cells (FIG. 6B)

The control group for the metastatic ovarian cancer cells (A2780) consisted of three sample-sets similar to the HUVECs. The MagSiNs themselves were not toxic to the A2780 in the presence or absence of any magnetic fields. For the A2780, 20 nM Dox in its free form killed >20% cells. 20 nM Dox after release from MagSiNs resulted in no significant cell death. 83% cell death was seen after exposure to 500 nM Dox in its free form. 53% cell death was observed after 500 nM Dox was released from MagSiNs indicating reduced Dox anti-cancer efficacy after release from MagSiNs.

### PC-3 Prostate Cancer Cells (FIG. 6C)

The control group for the metastatic prostate cancer cells (PC-3) consisted of three sample-sets similar to the HUVEC. For PC-3, 20 nM Dox in its free form or after release from MagSiNs resulted in >20% cell death. 100% cell death was seen after exposure to 500 nM Dox in its free form. 47% cell death was observed after 500 nM Dox was released from MagSiNs, indicating reduced Dox anti-cancer efficacy after release from MagSiNs. The MagSiNs themselves were not toxic to the PC-3 cells in the presence or absence of any magnetic fields. Interestingly, 20 nM Dox·HCl in its free-form or when released from an equivalent dose of Dox-MagSiNs had similar anticancer activity. The similar anti-cancer efficacy of Dox·HCl from the two Dox·HCl formulations at the low dose (20 nM), but the dramatic difference in anti-cancer efficacy at the high dose (500 nM), might be indicative that the differences in the instantaneous dose of Dox·HCl released from Dox-MagSiNs becomes more pronounced at the higher dose. The difference in instantaneous dose exposure is ˜16 nM vs. 20 nM for the low dose and 400 nM vs. 500 nM for the higher dose, based on the release kinetics from Table 2.

### MDA-MB-231 Triple-Negative Breast Cancer Cells (FIG. 6D)

The control group for the metastatic triple-negative breast cancer cells (MDA-MB-231) consisted of three sample-sets similar to the HUVECs. The MagSiNs themselves were not toxic to the MDA-MB-231 cells in the presence or absence of any magnetic fields. For MDA-MB-231, 20 nM Dox in its free form or after release from MagSiNs resulted in insignificant cell death. 4% cell death was observed after exposure to 500 nM Dox in its free form. Approximately 10% cell death was observed after 500 nM Dox was released from MagSiNs, indicating increased Dox anti-cancer efficacy after release from MagSiNs. However, overall MDA-MB-231 cells were significantly chemoresistant to the dosages of Dox·HCl that were administered in free-form or as Dox-MagSiNs. This was not surprising considering the highly-efficient chemo-resistant mechanisms present in MDA-MB-231 cells.

The HUVEC results demonstrated that the Dox did not lose its activity after release from the Dox-MagSiNs. The significant difference in anti-cancer activity of 500 nM Dox·HCl in its free-form and from Dox-MagSiNs might be due to instantaneous exposure of the cancer cells to the free form of Dox·HCl, as opposed to the drug released from the Dox-MagSiNs, which is 80% of the equivalent dose at 30 min post AC magnetic stimulation. However, the advantage here is that unlike standard chemotherapy, due to the non-cytotoxic nature of Dox-MagSiNs, there is the possibility of attacking the cancer cells with multiple doses of Dox-MagSiNs, which is not possible with free Dox·HCl due to the indiscriminate toxicity of Dox·HCl in its free form. Therefore, it is possible to increase the therapeutic window of standard chemotherapeutics like Dox·HCl by utilizing MagSiNs as drug carriers.

Overall, the MagSiNs themselves or 500 nM Dox-MagSiNs were not toxic to HUVEC cells or any of the tested cancer cells. HUVEC sensitivity to Dox-induced toxicity is well known. Additionally, the membrane potential of HUVECs is also depolarized similar to cancer cells. However, since Dox from Dox-MagSiNs did not have enhanced anticancer efficacy against the cancer cells, it is reasonable that the observed HUVEC cell death was due to that cell line's increased sensitivity to Dox·HCl and not due to enhanced uptake of Dox-MagSiNs in a magnetic field. PC-3 and A2780 showed statistically significant cell death after exposure to 500 nM Dox·HCl released from Dox-MagSiNs. 100% more MDA-MB-231 cells were killed with Dox·HCl released from Dox-MagSiNs in comparison to free Dox·HCl. However, there was still significant chemoresistance of the MDA-MB-231 cells to Dox·HCl, pointing to the need for a combinatorial treatment to nullify the chemoresistant mechanisms and re-sensitize the cells to Dox treatment. One of the biggest advantages of Dox-MagSiNs is that they negate non-specific toxicity from Dox·HCl, as was evident with the 500 nM Dox-MagSiNs-treated HUVECs having statistically similar viability to untreated HUVEC cells control groups. This implies that Dox-MagSiNs can be systemically delivered to avoid off-target Dox·HCl toxicity, and the full dose of Dox·HCl can be delivered near instantaneously to the cancer by using a localized alternating magnetic field to trigger the release of the Dox·HCl. To elucidate the mode of interaction of the Dox-MagSiNs with the different cells, co-localization studies were performed by staining sub-cellular features and imaging using confocal microscopy.

### Example 8

### Co-Localization Assays for Dox-MagSiNs in Cells

Lysosome co-localization assays were performed to elucidate a probable cause for the varied response of the different cancer cells to Dox·HCl released from MagSiNs. Co-localization assay for MagSiNs in cells was performed. The silica-shell of MagSiNs was volume loaded with Rhodamine-B red fluorescent dye. Lysosomes were stained with lysoview-green. Nucleus was stained blue with DAPI. The cell membrane was imaged using phase-contrast illumination. Co-localization was assessed in the presence and in the absence of a 27-35 Gauss, permanent magnetic field.

### HUVEC Control Cells

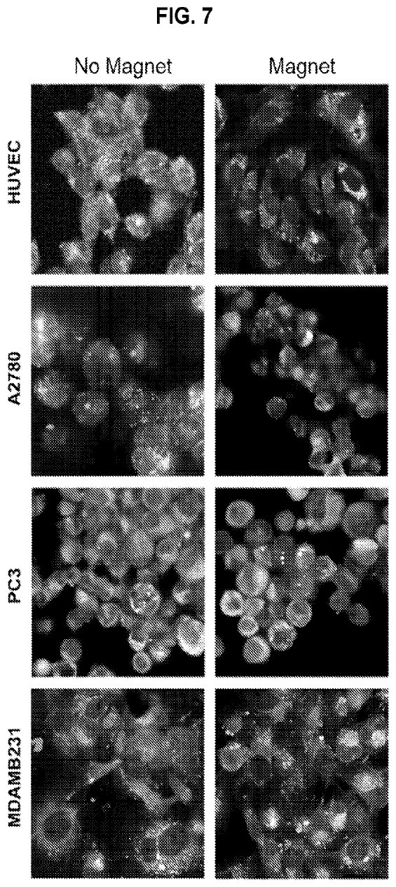

FIG. 7 shows no dependence on the external magnetic field on the co-localization of the MagSiNs extracellularly or intra-cellularly for HUVECs. Lysotracker dye indicated 3-5 lysosomes per cell. Co-localization study indicated that the number of MagSiNs clusters associated with HUVECs or internalized was similar regardless of the presence or absence of the weak magnetic field. Because of their stem-cell-like origin from the umbilical veins, HUVECs have depolarized membrane potentials ranging from −11 mV to −17 mV, similar in range to the cancer cells tested here (Table 3). However, the Young's modulus of their membrane (10-11 kPa) is 20-fold to 40-fold higher than the cancer cell lines tested here. The co-localization study results indicate that the HUVEC cell death when exposed to 20 nM or 500 nM Dox·HCl either as free Dox·HCl or from Dox-MagSiNs is due to the well documented sensitivity of HUVECs to Dox·HCl and not because of enhanced uptake of Dox-MagSiNs.

### A2780 Ovarian Cancer Cells

FIG. 7 shows well-defined and distinct lysosomes in the absence of a magnetic field for A2780 cells. In the presence of a magnetic field, the MagSiNs were clustered to form 1-2 μm structures. Yellow fluorescence signal that indicated co-localization of MagSiNs with lysosomes accounted for less than 10% of the MagSiNs signal. Majority red fluorescence signal from MagSiNs indicated the lack of co-localization of the MagSiNs with lysosomes in the presence of a magnetic field. There was also a lack of distinct lysosomes in the cells in the presence of MagSiNs and a magnetic field. There is a distinct possibility that the >1 μm sized Dox-MagSiNs structures were able to disrupt lysosomes in the presence of the magnetic field, which would explain the lack of distinct lysosomes. The Pearson's coefficients for co-localization with and without magnetic field were 0.89 and 0.92, respectively, which also concurred with the fact that there was only a slight increase in the co-localization signal between the green and red channel. While there was significant cell death in the presence of 500 nM dose of Dox·HCl as a free formulation or from 500 nM Dox·MagSiNs, the Dox delivered from Dox-MagSiNs resulted in 50-55% cancer cell death as opposed to 80-85% cell death observed with free Dox formulation. The fact that only 80% of Dox is released instantaneously from 500 nM Dox-MagSiNs, combined with about 10% of the Dox-MagSiNs being sequestered in lysosomes might account for the discrepancy in the efficacy of Dox released from Dox-MagSiNs in comparison to Dox used as a free formulation.

### PC-3 Prostate Cancer Cells

FIG. 7 shows well-defined and extensive lysosomes for PC-3 cells. As with A2780, sequestration of the Dox-MagSiNs in PC-3 lysosomes was observed intracellularly in the absence of a magnetic field as evidenced by the dense overlays of the fluorescent signal of the MagSiNs with the lysosomes resulting in a strong yellow fluorescent signal. In the presence of a magnetic field, Dox-MagSiNs were clustered in cells, although they were not majority co-localized with the lysosomes and some well-delineated lysosomes remained. Approximately 19% of the fluorescent signal from Dox-MagSiNs was co-localized with the lysosomes. Furthermore, unlike A2780 cells, only ˜8% of the Dox-MagSiNs clusters incubated with PC-3 cells in the presence of a magnetic field were >1 μm. It is possible that similar to A2780 cells, that MagSiNs in PC-3 cells were sequestered into lysosomes, but that the >1 μm MagSiNs clusters were able to disrupt the lysosomes in the magnetic field similar to data from previous studies. The Pearson's coefficient for co-localization with and without magnetic field dropped from 0.95 to 0.86, which would support the theory that MagSiNs in a magnetic field can disrupt lysosomes. While there was significant cell death in the presence of 500 nM dose of Dox·HCl as a free formulation or from 500 nM Dox·MagSiNs, the Dox delivered from Dox-MagSiNs resulted in ˜47% cancer cell death as opposed to >95% cell death observed with free Dox formulation. The reduced anti-cancer efficacy data of Dox·HCl released from Dox-MagSiNs correlates well with the co-localization data that indicated lysosomal sequestration of ˜20% of the MagSiNs, which would imply that the instantaneous Dox·HCl dose released from Dox-MagSiNs will not be equivalent to 500 nM of free Dox·HCl.

### MDA-MB-231 Triple-Negative Breast Cancer Cells

FIG. 7 shows dense packing with lysosomes for MDA-MB-231 cells. In the absence of a magnetic field, the MagSiNs were barely co-localized with the cells (Pearson's coefficient=0.77). In the presence of a magnetic field >85% of the MagSiNs, fluorescent signals were heavily co-localized with the lysosomes in the overlay, indicating efficient sequestration of the MagSiNs in the lysosomes (Pearson's coefficient=0.87). More than 70% of the >1 μm MagSiNs clusters were also co-localized with the lysosomes. While there appeared to be a slight increase of 10% cancer cell death in the presence of 500 nM Dox. MagSiNs in comparison to the 4% cancer cell death for a 500 nM dose of Dox·HCl; unlike PC-3 or A2780, it appears that even if the MagSiNs can efficiently disrupt the lysosomes in the presence of a magnetic field, there are enough number of lysosomes to re-sequester the MagSiNs. The co-localization study results would indicate the need for either using sufficiently large numbers of MagSiNs whose combined volume would overwhelm the combined volume capacity of lysosomes per cell or to modify MagSiNs with surface chemistry that would make them impossible to be sequestered in lysosomes. Therefore, although MagSiNs were efficiently internalized into MDA-MB-231, their sequestration in the lysosomes would indicate that the chemoresistant mechanisms of MDA-MB-231 were as efficient in negating the effect of 500 nM of free Dox as they were in negating Dox released from Dox-MagSiNs, based on the results of the cell viability studies.

The difference in membrane physical properties of cancer cells was exploited for chemotherapeutics delivery by Dox-MagSiNs by applying a magneto-electric charge and force that is above the threshold required to nanoporate the abnormal cells, but below the threshold required to nanoporate normal cells. Three different techniques were tested to load the Dox·HCl on MagSiNs and it was determined that covalent immobilization of Dox·HCl on MagSiNs formed the most stable nanocarriers with near zero-order drug release kinetics. It was determined that an external magnetic field in the range of 25-50 Gauss at the cell membrane interface generates a magneto-electric charge and force on the Dox-MagSiNs that allows it to permeabilize the cancer cells and not the healthy cells (e.g., HUVECs). Preliminary studies confirmed that Dox was released on-demand from Dox-MagSiNs using an alternating magnetic field of 25-50 Gauss with a frequency range of 50-100 Hz, which then proceeded to significantly kill (˜50%) two out of the three cancer cells (A2780 and PC-3 cells) that were tested against, in a dose-dependent manner. When the Dox·HCl was released from the Dox-MagSiNs, the preferential accumulation of Dox-MagSiNs in cancer cells enhanced anti-cancer activity of Dox·HCl against two (PC-3 and A2780 cells) out of the three cancer cell lines tested. The enhanced sensitivity of neo-vasculature such as those associated with cancer cells to existing chemotherapeutics resulted in 100% cell death of HUVECS once Dox·HCl was released from Dox-MagSiNs. This was despite the fact that there was no significant internalization of the Dox-MagSiNs by HUVECs in the low magnetic-field. Dox-MagSiNs also killed 50% more triple-negative breast cancer MDA-MB-231 cells in comparison to the same dosage of free Dox·HCl.

Three important outcomes of this study were that (a) the drug-carrying magneto-electric nanocarriers (Dox-MagSiNs) were completely biocompatible; (b) a localized alternating magnetic field may be used to release the Dox·HCl from the Dox-MagSiNs in the vicinity of the tumor to negate any off-target toxicity associated with systemic delivery of drug molecules; and (c) the rapid release kinetics of the payload from MagSiNs in the presence of an external alternating magnetic field ensures >50% cancer cell killing efficacy. This concept is novel as the surface electrical potential (the zeta-potential) of the MagSiNs is being tuned to match the membrane potential of cancer cells to increase interaction with the cells and to ensure selective nanoporation into pliant cancer cell membranes and not the order-of-magnitude stiffer healthy cell membranes. While nanoporation into cancer cells resulted in >50% cell death of PC-3 and >47% cell death of A2780 cells at the high-dose (500 nM) of Dox·HCl, toxicity from the Dox·HCl released from Dox-MagSiNs also resulted in HUVEC cell death. The ability of the Dox-MagSiNs in a localized alternating magnetic field to destroy cancer cells and associated tumor vasculature is promising as this can lead to enhanced permeability and retention (EPR) effect. The MRI contrast properties along with fluorescence signal from the MagSiNs may also aid in image-guided localization to the tumor. It is also advantageous that the Dox-MagSiNs do not need to be tagged with targeting molecules as some of the targeting labels are known to be systemically toxic themselves (e.g., Trastuzumab). Finally, since the Dox-MagSiNs are bio-inert, the cancer cells can be exposed to multiple doses, leading to higher cumulative doses of Dox-MagSiNs, in comparison to free Dox·HCl. The systemic biocompatibility is a major step forward in increasing the tolerable total dose of chemotherapeutic that is currently allowed, thus increasing the therapeutic window and anti-cancer efficacy of an important class of existing chemotherapeutics such as anthracyclines, while essentially eliminating their harmful side-effects due to off-target activity.

### Example 9

### MagSiNs Conjugated to Other Chemotherapeutic Drugs

### Migration/Invasion Assay

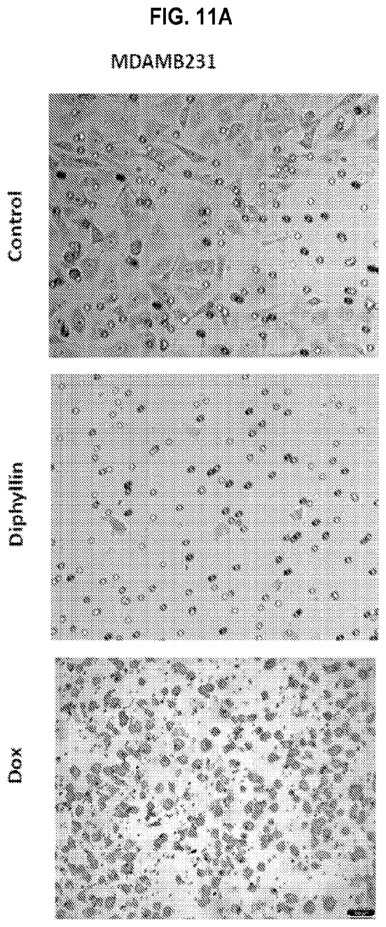

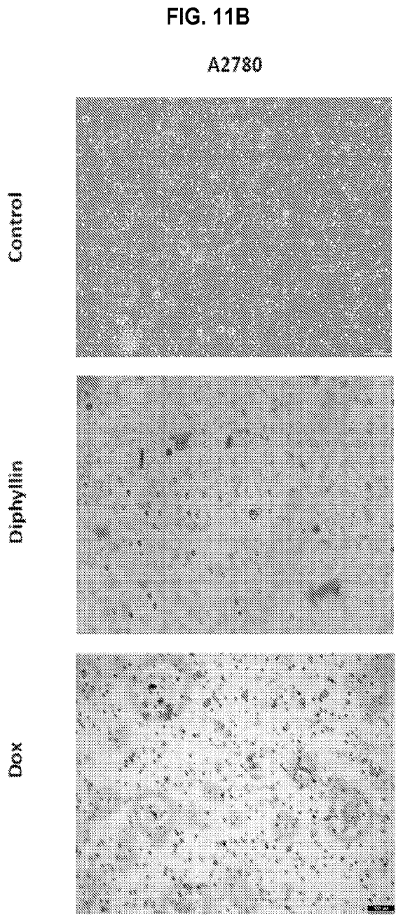

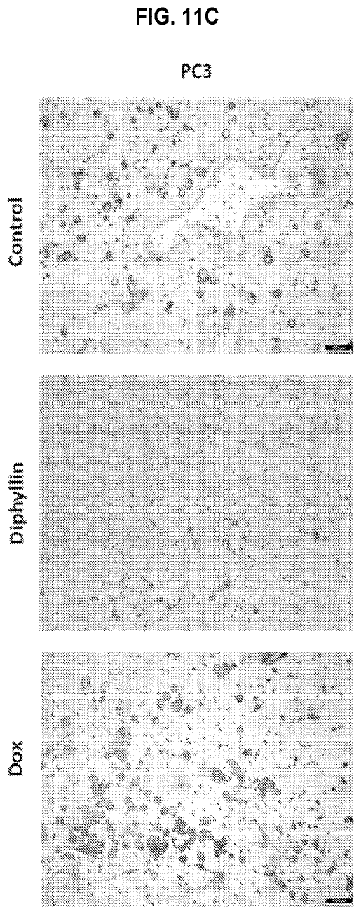

A transwell migration assay was used to test the effect of doxorubicin or diphyllin released from MagSiNs on the ability of PC-3 prostate cancer cells, A2780 ovarian cancer cells, and MDA-MB-231 triple negative breast cancer cells to migrate. Diphyllin is a drug that inhibits vacuolar ATPase pumps (V-ATPases) used by cancer cells to degrade surrounding collagen and acidify their extracellular environment which in turns allows them to metastasize. Dox·HCl treatment alone was found to kill cancer cells but also stimulated them to migrate. In combination with diphyllin, which prevents cell migration, Dox·HCl was able to kill a greater number of cancer cells. Diphyllin (40 nM) released from the MagSiNs was also found to inhibit the transwell migration of all cancer cells tested (Table 5; FIG. 11A-C). These results indicated that diphyllin released from MagSiNs may be used complementarily at a low dose (<40 nM) to prevent tumor metastases during chemotherapeutic tumor treatment. In addition, MagSiNs loaded with both diphyllin and doxorubicin in combination at a higher dose (>500 nM) may synergistically kill cancer cells and prevent cancer cell metastasis.

TABLE 5
Transwell membrane migration/invasion assay of cancer cells
treated with MagSiNs loaded with diphyllin or doxorubicin

| | % Cell Migration (mean ± st. dev.) | | |
|---|---|---|---|
| Treatment | MDA-MB-231 | PC-3 | A2780 |
| Diphyllin-MagSiNs | 28.0 ± 15.8% | 69.2 ± 15.2% | 40.2 ± 3.1% |
| Dox-MagSiNs | 100.0 ± 0.0% | 100.0 ± 0.0% | 79.3 ± 8.0% |

### Live/Dead Cell Viability Assays

A unidirectional magnetic field of 30-50 Gauss and an alternating magnetic field of 100 Hz, 30-50 Gauss were used for these experiments. The alkyne-modified diphyllin was conjugated to azide-modified MagSiNs using copper catalyzed CLICK chemistry to create Diph-MagSiNs. The amine on Dox·HCl was conjugated to acid anhydride-modified MagSiNs to create Dox-MagSiNs. Mass spectrometry was used to determine drug loading on MagSiNs. For co-delivery of doxorubicin and diphyllin, the required dosage of Diph-MagSiNs with respect to the diphyllin dose and the required dosage of Dox-MagSiNs with respect to Dox·HCl dose were mixed together and delivered as a single bolus.

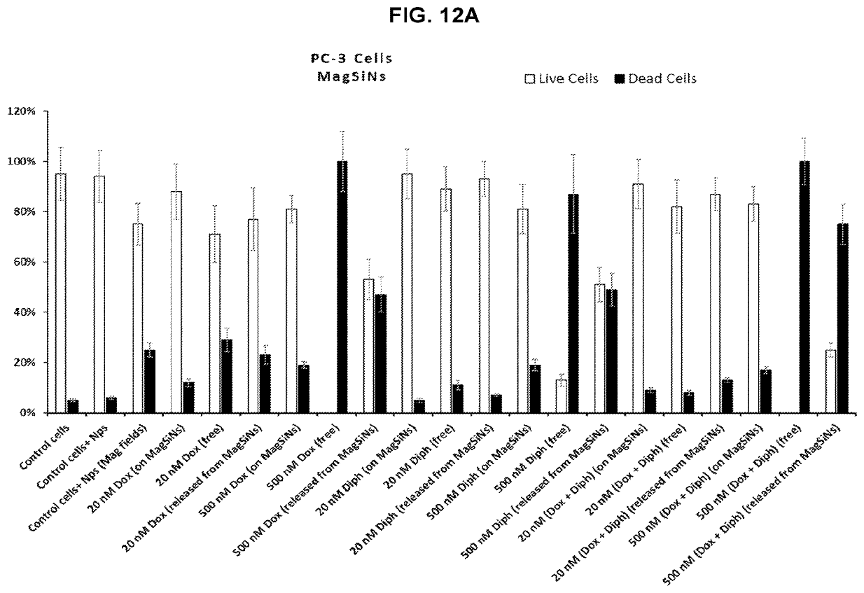

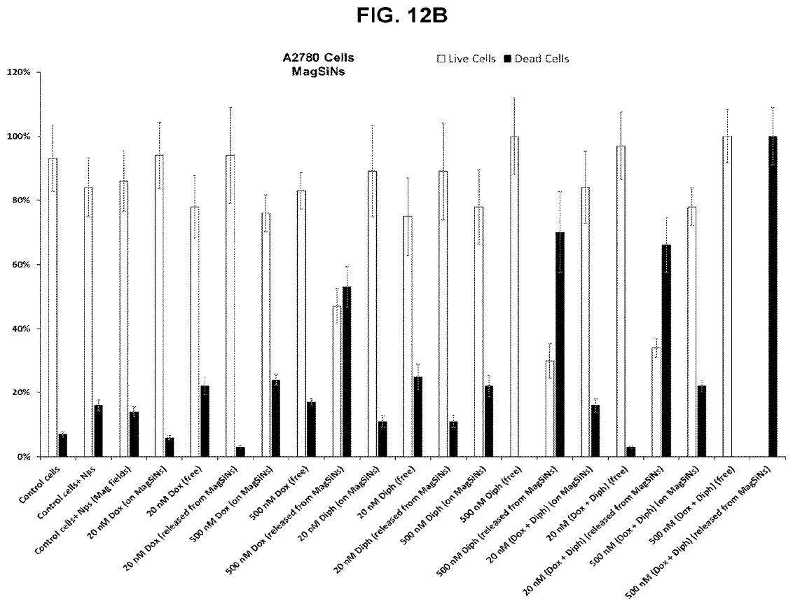

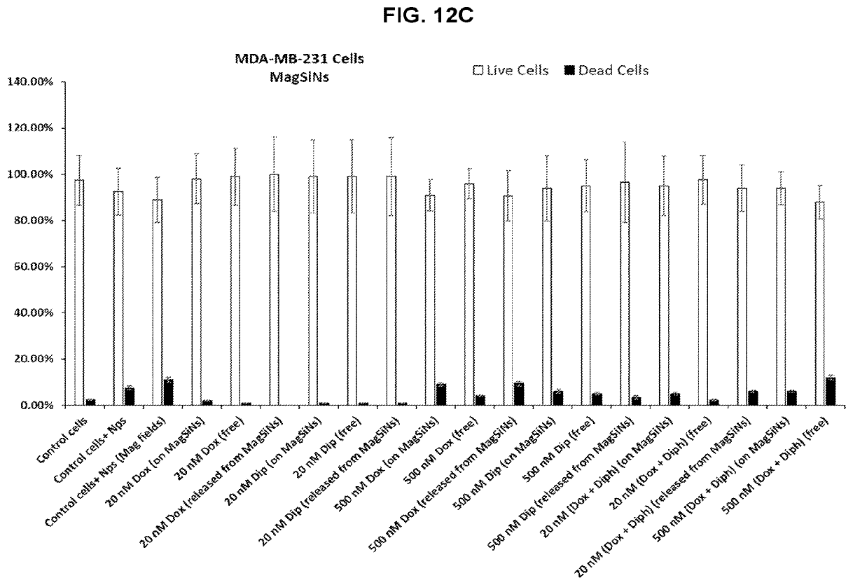

FIG. 12A-C show live/dead cell assay results of PC-3 prostate cancer cells (FIG. 12A), A2780 ovarian cancer cells (FIG. 12B), and MDA-MB-231 triple negative breast cancer cells (FIG. 12C) administered MagSiNs conjugated to hydrophilic doxorubicin, hydrophobic diphyllin, or a combination of both chemotherapeutic drug molecules.

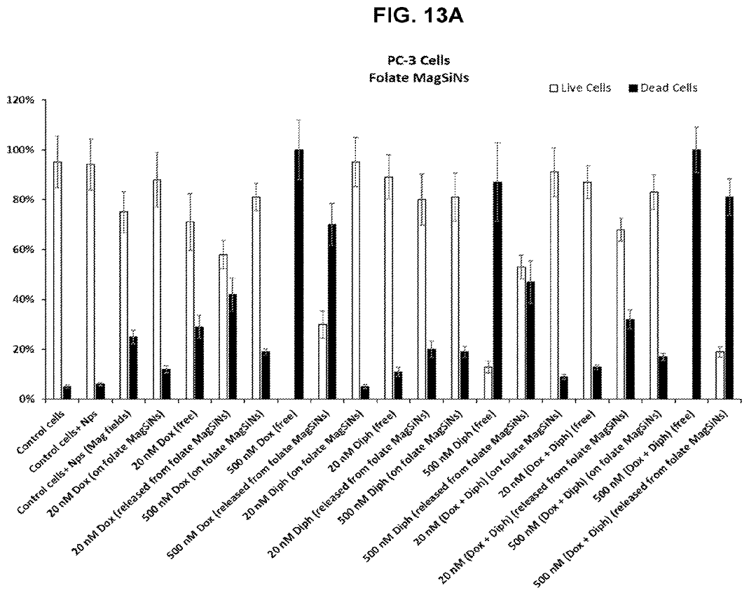

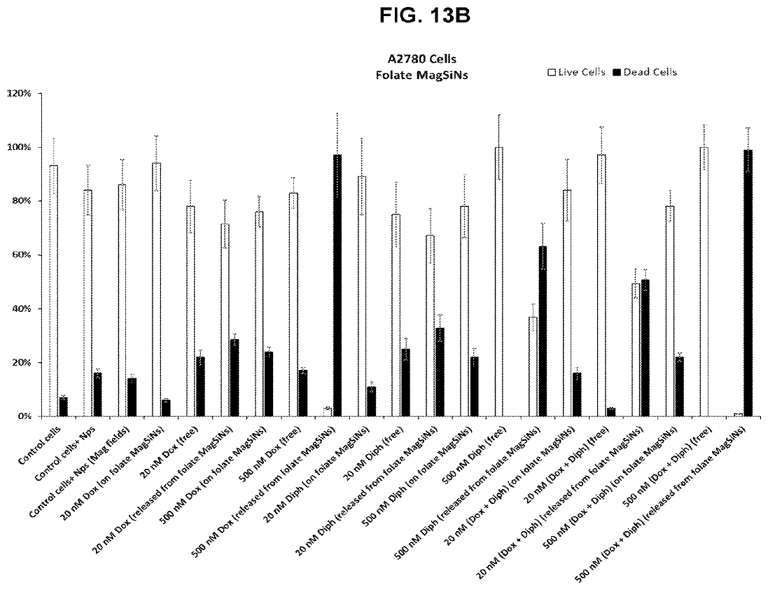

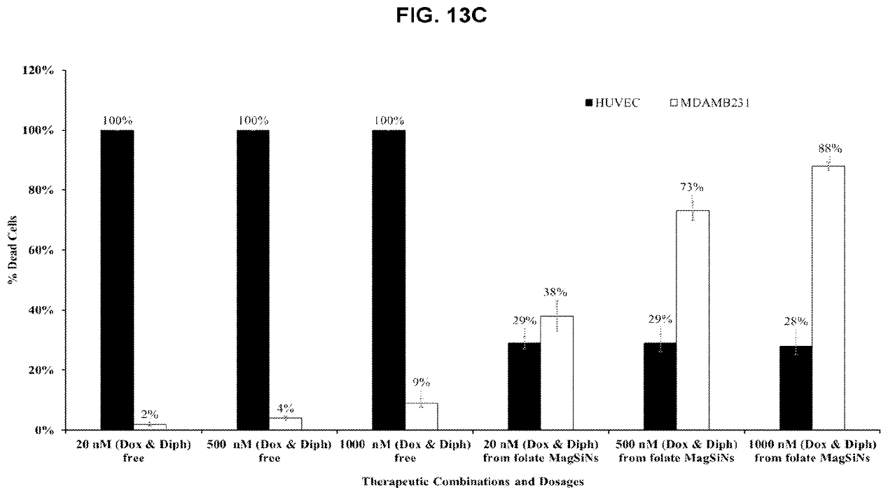

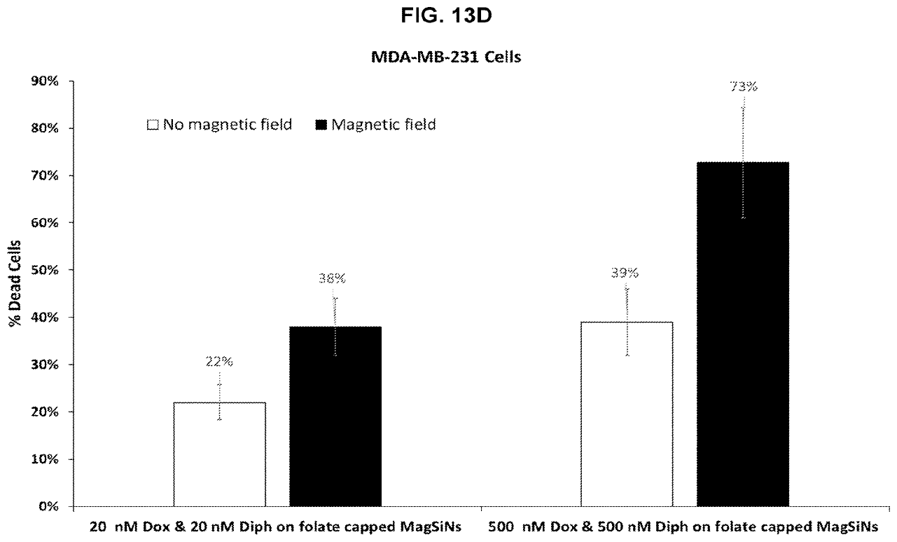

Furthermore, FIG. 13A-B show live/dead cell assay results of PC-3 prostate cancer cells (FIG. 13A) and A2780 ovarian cancer cells (FIG. 13B) administered folate conjugated MagSiNs conjugated to hydrophilic doxorubicin, hydrophobic diphyllin, or a combination of both chemotherapeutic drug molecules. In addition, the viability assay was also used to test the drug-loaded folate MagSiNs in HUVEC cells and MDA-MB-231 breast cancer cells (FIG. 13C). The combined formulations of [Dox+Diph] delivered on folate MagSiNs were 10× to 19× more effective in killing the MDA-MB-231 cells than the free-drug formulations of the same dosages (FIG. 13C). These studies also demonstrated the reduced toxicity of the MagSiNs in control healthy cells (e.g., HUVEC cells). It was also found that enhanced anti-cancer efficacy of the folate MagSiNs is dependent on the external magnetic field applied (FIG. 13D). The external magnetic field helps to enhance cancer cell permeation and maximize the intracellular drug delivery from MagSiNs loaded with [Dox+Diph] (FIG. 13D). Specifically, a combined formulation of [Dox+Diph] loaded on folate conjugated MagSiNs was found to be twice as effective in killing the cancer cells when an external magnetic field was applied compared to when no magnetic field was applied (FIG. 13D).

### Example 10

In Vivo Mice Studies with Fluorescent MagSiNs

Triple negative MDA-MB-231 breast cancer cells (MDA-231-RFP) were injected in the right rear flank of Foxn1^{nu/nu} athymic mice (Jackson Labs). Specifically, 500,000 to 1,000,000 MDA-231-RFP cells were suspended in sterile PBS and injected in each mouse. The MDA-MB-231 tumors were allowed to grow to at least 5 mm in diameter over 6 weeks. The mice with tumors were then injected with 200 μL of 10 mg/mL Dylight750 fluorescent MagSiNs using tail vein injections. The MagSiNs were surface functionalized with 3-(triethoxysilyl) propylsuccinic anhydride (Gelest). The acid anhydride was conjugated to Doxorubicin. HCl (Dox·HCl). The total dose was 50 mg/kg of Dox-MagSiNs per mouse administered intravenously.

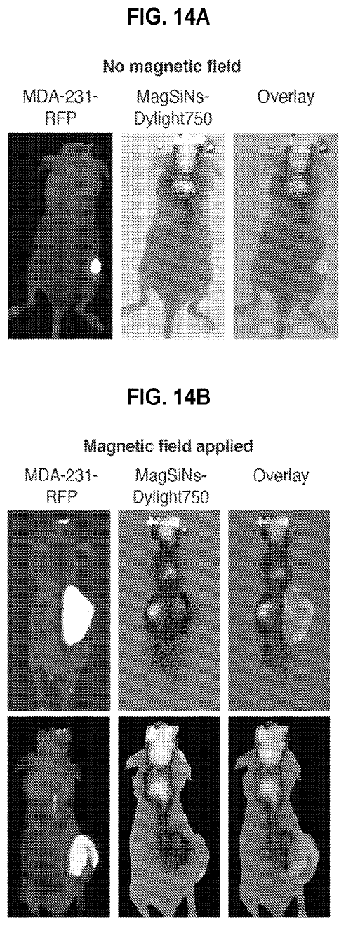

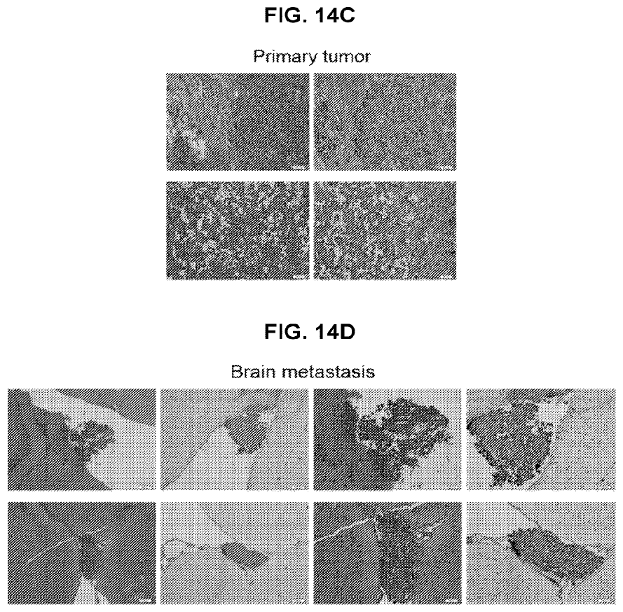

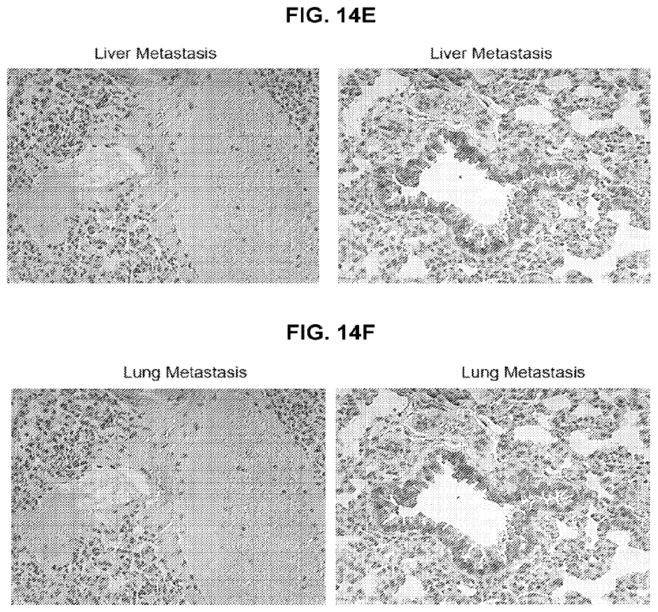

The mice were then placed in cages placed above an array of permanent magnets. The mice were either not exposed to a magnetic field (FIG. 14A) or exposed to a 30-40 Gauss whole body permanent magnetic field (FIG. 14B) for 18-24 hr before being removed from the cage and imaged using in vivo fluorescence in two channels-RFP for tumor cells and near-IR to track the MagSiNs. The MagSiNs were found to be localized to the primary tumor tissue and metastasized sites in the mice exposed to the whole-body magnetic field (FIG. 14B). The mice were then sacrificed and their organs, tumor, and blood were collected for analysis.

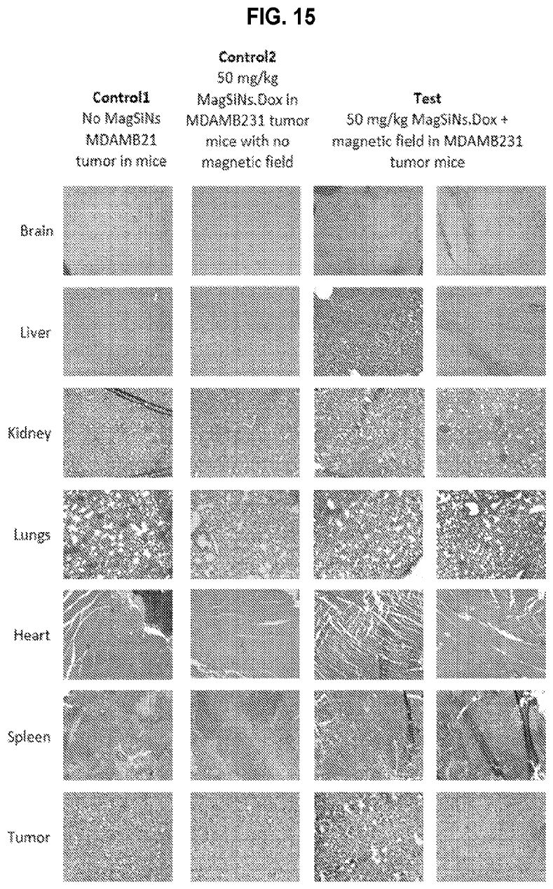

Histology (IHC) was performed on the collected tissues to observe any metastases (FIG. 14A-F) or inflammation (FIG. 15) in organs. FIG. 14A-F show that IHC for the marker Cytokeratin-8 clearly confirmed that injected MDA-MB-231 cells created primary tumors in mice and metastasized to the brain, lungs, and liver. IHC also confirmed that Dox-MagSiNs were not randomly bioaccumulating in different tissues in vivo when a magnetic field was applied and were targeting the primary tumor and other sites to which the cancer cells had metastasized. FIG. 15 shows that there was no inflammation in mice injected with the Dox-MagSiNs in comparison to the control mice which were not injected with Dox-MagSiNs. This confirmed that the Dox-MagSiNS were biocompatible similar to MagSiNs alone and have the ability to carry Doxorubicin without inducing systemic toxicity in vivo.

Additional in vivo dosing studies were also conducted with different drug loading on MagSiNs. For diphyllin loading, 11.3 mg of MagSiNs was conjugated to 9.996 mg of diphyllin at 99.96% conjugation efficiency. Liquid chromatography-mass spectrometry (LC-MS) was used to confirm diphyllin loading on MagSiNs. The conjugation was through an ester bond between an-OH group on diphyllin and —COOH groups on the silanes of the MagSiNs. For doxorubicin loading, 11.3 mg of MagSiNs was conjugated to 1.752 mg of Dox·HCl (0.16 mg of Dox·HCl/1 mg of MagSiNs). A dose of up to about 50 mg/kg of MagSiNs was found to be biocompatible in mice. A dosing regimen of about 5 mg/kg of Dox·HCl per week is a standard maximum dose of Dox·HCl in mice. This roughly translates to about 0.1 mg of Dox·HCl per mouse, which is equivalent to about 0.625 mg of Dox-MagSiNs per mouse (31.25 mg Dox-MagSiNs/kg of mouse).

### Example 11

Additional Studies of Folate Conjugated MagSiNs Loaded with Chemotherapeutics

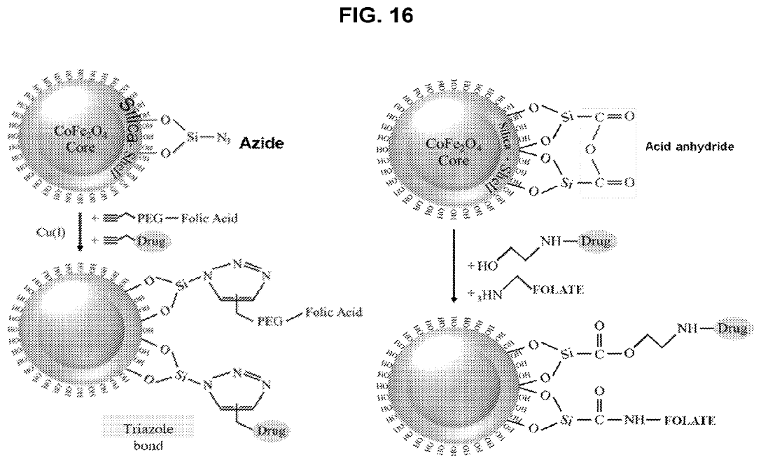

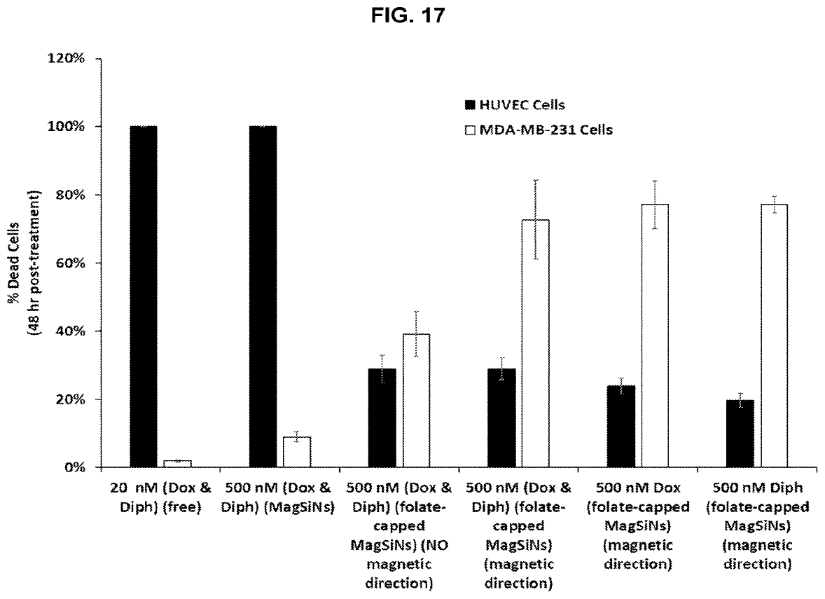

Folate-PEG-conjugated MagSiNs loaded with doxorubicin and/or diphyllin were prepared (FIG. 16). These folate-PEG-conjugated MagSiNs loaded with drug were found to kill MDA-MB-231 breast cancer cells at a much higher level compared to normal control HUVEC cells, even after exposure to an alternating magnetic field to trigger drug release (FIG. 17). The folate-PEG-conjugated MagSiNs loaded with drugs (doxorubicin and/or diphyllin) were also found to kill PC-3 prostate cancer cells and A2780 ovarian cancer cells in a low magnetic field. The concentration range of drugs tested was 20 nM-500 nM. Normal HUVEC control cells showed resistance to the folate-PEG-conjugated MagSiNs loaded with drugs and had higher viability than the cancer cells.

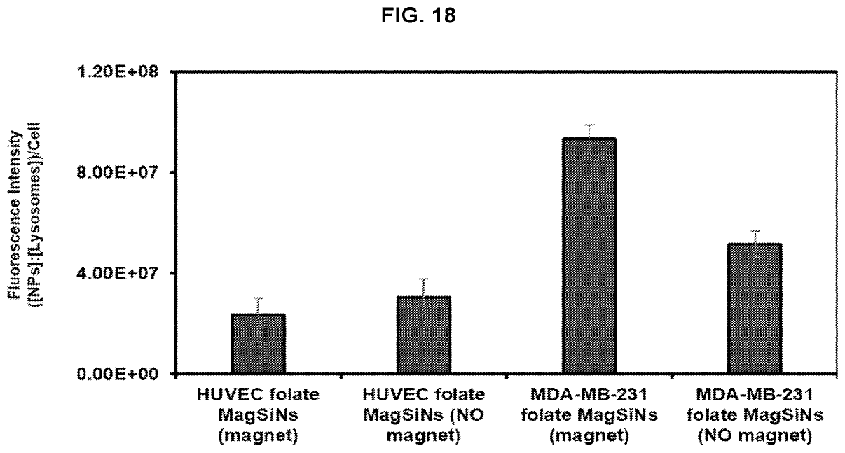

In addition, Lysotracker assay results indicated that folate-conjugated drug-MagSiNs were highly efficient in disrupting lysosomes in MDA-MB-231 breast cancer cells in the presence of an alternating magnetic field (FIG. 18). For control HUVEC cells, there was statistically no difference in the fluorescence intensity ratio between cells exposed to folate-conjugated drug-MagSiNs in the presence or absence of an alternating magnetic field (FIG. 18). For the MDA-MB-231 breast cancer cells, there was a significant difference between cells exposed to folate-conjugated drug-MagSiNs in the presence of a magnetic field as compared to in the absence of a magnetic field (FIG. 18). These results help to explain why the folate-conjugated drug-MagSiNs were highly efficient in killing MDA-MB-231 cells when they were later exposed to an alternating magnetic field. The fact that folate-conjugated drug-MagSiNs did not even enter HUVEC cells at a high level helps explain why they did not kill the HUVEC control cells after exposure to an alternating magnetic field. This is likely because in control HUVEC cells, the PEG-folate drug-MagSiNs are in a neutral pH extracellular matrix environment and the PEG prevents the diffusion of drugs away from the MagSiNs after exposure to an alternating magnetic field, thus maintaining HUVEC cell viability compared to cancer cells.

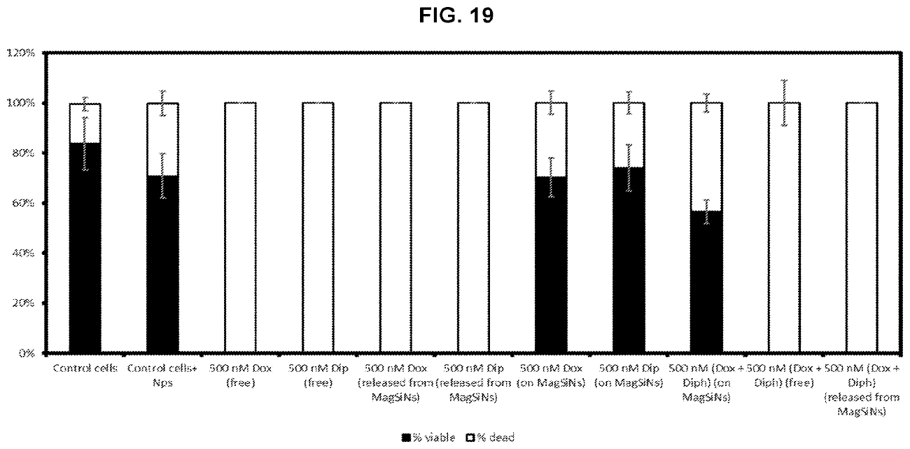

Furthermore, HUVEC control cells exposed to non-folate-conjugated drug-MagSiNs were killed immediately after exposure to an alternating magnetic field to trigger drug release, similar to treatment with free doxorubicin or diphyllin (FIG. 19). The non-folate-conjugated drug-MagSiNs were only found to be biocompatible in the HUVEC cells in the absence of an alternating magnetic field (FIG. 19).

What is claimed:

1. A magnetoelectric nanoparticle composition for targeted and controlled drug release, the composition comprising:
a cobalt ferrite (CoFe₂O₄) magnetic nanoparticle core;
a fused silica shell; and
one or more therapeutic agents covalently conjugated to the fused silica shell.

2. The composition of claim 1, wherein the one or more therapeutic agents comprise one or more chemotherapeutic drugs comprising doxorubicin or diphyllin.

3. The composition of claim 1, further comprising a polyethylene glycol (PEG)-linked folate or folic acid molecule covalently conjugated to the fused silica shell.

4. The composition of claim 1, wherein the cobalt ferrite magnetic nanoparticle core with the fused silica shell has a diameter of about 1 nm to about 18 nm.

5. The composition of claim 4, wherein the cobalt ferrite magnetic nanoparticle core with the fused silica shell has a diameter of about 4 nm to about 8 nm.

6. The composition of claim 1, wherein the one or more therapeutic agents are covalently conjugated to the fused silica shell through silanization with a succinic acid anhydride moiety on the fused silica shell.

7. A system for targeted and controlled drug release, the system comprising:
a magnetoelectric nanoparticle composition comprising:
a cobalt ferrite (CoFe₂O₄) magnetic nanoparticle core;
a fused silica shell; and
one or more therapeutic agents covalently conjugated to the fused silica shell; and
an alternating current electromagnetic field source.

8. The system of claim 7, wherein the alternating current electromagnetic field source comprises an electromagnet coupled to a sinusoidal alternating current generator.

9. The system of claim 7, wherein the alternating current electromagnetic field source generates a magnetic field strength of about 20 Gauss to about 60 Gauss.

10. The system of claim 9, wherein the alternating current electromagnetic field source generates a magnetic field strength of about 25 Gauss to about 50 Gauss.

11. A method for treating a subject having cancer or at risk of developing cancer, the method comprising:
administering to the subject a therapeutically effective amount of a magnetoelectric nanoparticle composition comprising:
a cobalt ferrite (CoFe₂O₄) magnetic nanoparticle core;
a fused silica shell; and
one or more therapeutic agents covalently conjugated to the fused silica shell; and
applying an external alternating current electromagnetic field to the subject, thereby releasing the one or more therapeutic agents from the administered magnetoelectric nanoparticle composition.

12. The method of claim 11, wherein the one or more therapeutic agents comprise one or more chemotherapeutic drugs comprising doxorubicin or diphyllin.

13. The method of claim 11, wherein the external alternating current electromagnetic field comprises a magnetic field strength of about 20 Gauss to about 60 Gauss.

14. The method of claim 13, wherein the external alternating current electromagnetic field comprises a magnetic field strength of about 25 Gauss to about 50 Gauss.

15. The method of claim 11, wherein the external alternating current electromagnetic field is applied to the subject for a period of time of about 1 minute to about 48 hours.

16. The method of claim 11, wherein the external alternating current electromagnetic field is applied to the subject at a frequency of about 50 Hz to about 150 Hz.

17. The method of claim 11, wherein the one or more therapeutic agents are released from the magnetoelectric nanoparticle composition at a primary tumor site, a metastatic tumor site, or a combination thereof in the subject.

18. The method of claim 11, wherein the therapeutically effective amount of the magnetoelectric nanoparticle composition is about 5 mg/kg to about 100 mg/kg.

19. The method of claim 11, wherein the therapeutically effective amount of the magnetoelectric nanoparticle composition is administered using a dosing regimen comprising a single dose or a plurality of doses.

20. A method of making a magnetoelectric nanoparticle composition for targeted and controlled release of doxorubicin, the method comprising:
mixing a magnetoelectric nanoparticle comprising a cobalt ferrite (CoFe₂O₄) magnetic nanoparticle core and a fused silica shell with (3-Triethoxysilyl) propylsuccinic anhydride to create a silanized magnetoelectric nanoparticle; and
reacting the silanized magnetoelectric nanoparticle with doxorubicin hydrochloride to create the magnetoelectric nanoparticle composition.

21. The method of claim 20, wherein the (3-Triethoxysilyl) propylsuccinic anhydride is dispersed in a mixture of ethanol and water.

22. The method of claim 20, wherein reacting the silanized magnetoelectric nanoparticle with doxorubicin hydrochloride comprises incubating at a temperature of about 4° C. for a period of time of about 24 hours.

23. The method of claim 20, further comprising reacting the silanized magnetoelectric nanoparticle with a polyethylene glycol (PEG)-linked folate or folic acid molecule in dimethyl formamide to covalently conjugate the PEG-linked folate or folic acid molecule to the silanized magnetoelectric nanoparticle.

24. The method of claim 23, wherein reacting the silanized magnetoelectric nanoparticle with the PEG-linked folate or folic acid molecule comprises a first incubation at room temperature for about 3 hours, and a second incubation at about 4° C. for about 24 hours.
